(19) United States
(12) Patent Application Publication
(10) Pub. No.: US 2018/0305341 A1
(43) Pub. Date: Oct. 25, 2018

(54) IMIDAZOLINONE DERIVATIVES AS TRPM8 ANTAGONISTS

(71) Applicant: RAQUALIA PHARMA INC., Nagoya-shi, Aichi (JP)

(72) Inventors: Yuji SHISHIDO, Aichi (JP); Masashi OHMI, Aichi (JP)

(73) Assignee: RaQualia Pharma Inc., Nagoya-shi, Aichi (JP)

(21) Appl. No.: 15/758,932

(22) PCT Filed: Sep. 12, 2016
(86) PCT No.: PCT/JP2016/004133

§ 371 Date: Mar. 9, 2018

Related U.S. Application Data
(60) Provisional application No. 62/217,228, filed on Sep. 11, 2015.

(51) Int. Cl. C07D 403/12; C07D 403/10; C07D 471/04

(52) U.S. Cl. CPC C07D 403/12; C07D 471/04; C07D 403/10

(57) ABSTRACT

The present invention relates to imidazolinone derivatives of the formula (I) or a pharmaceutically acceptable salt thereof or a prodrug thereof, processes for their preparation, pharmaceutical compositions containing them and their use in the treatment of various disorders which are mediated via the TRPM8 receptor.

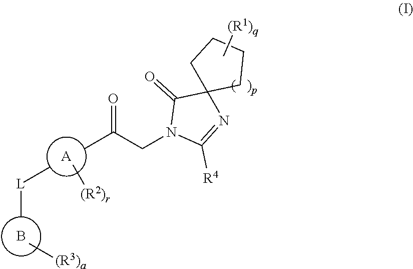

### TECHNICAL FIELD

This invention relates to imidazolinone derivatives that act as modulators of the TRPM8 receptor. The present invention also relates to processes for the preparation of novel imidazolinone derivatives and to their use in the treatment of a wide range of diseases, syndromes, and disorders, in particular for the treatment of inflammatory, pain and urological diseases or disorders.

### BACKGROUND ART

Transient receptor potential (TRP) channels are one of the largest groups of ion channels, and they are divided into 6 sub-families (TRPV, TRPM, TRPA, TRPC, TRPP and TRPML). TRP channels are cation-selective channels that are activated by a variety of physical (e.g., temperature, osmolarity, mechanical) and chemical stimuli. TRPM8 (transient receptor potential melastatin 8) is a member of TRP channel family. The receptor was cloned in 2002 (NPL 1; NPL 2) and it was found to be sensitive to cold temperature and menthol, and therefore named as cold menthol receptor-1 (CMR-1). TRPM8 can sense temperature changes in the range of both innocuous cold (15-28° C.) and noxious cold (<15° C.) as well as by chemical agents such as menthol and icilin.

TRPM8 is located on primary nociceptive neurons including A-delta and C-fibers and is also modulated by inflammation-mediated second messenger signals (NPL 3; NPL 4). The localization of TRPM8 on both A-delta and C-fibers may provide a basis for abnormal cold sensitivity in pathologic conditions wherein these neurons are altered, resulting in pain, often of a burning nature (NPL 5; NPL 6; NPL 7; NPL 8; NPL 9). Gauchan et al. reported that the expression of TRPM8 in the primary afferents was increased in oxaliplatin-induced cold allodynia model in mice (NPL 10). Oxaliplatin, a third-generation platinum-based chemotherapy drug, induces serious sensory neurotoxicity in patients, which is aggravated by exposure to cold. Recently, Glenmark group reported that the small molecular TRPM8 antagonists produced a dose-dependent inhibition of nocifensive paw licking in oxaliplatin-induced cold allodynia in mice (NPL 11).

Cold intolerance and paradoxical burning sensations induced by chemical or thermal cooling closely parallel symptoms seen in a wide range of clinical disorders and thus provide a strong rationale for the development of TRPM8 modulators as novel antihyperalgesic or antiallodynic agents. TRPM8 is also known to be expressed in the brain, odontoblasts, lung, bladder, gastrointestinal tract, blood vessels, prostate and immune cells, thereby providing the possibility for therapeutic modulation in a wide range of maladies.

International patent application WO 2006/040136 (PTL 1) purportedly describes substituted 4-benzyloxy-phenylmethylamide derivatives as cold menthol receptor-1 (CMR-1) antagonists for the treatment of urological disorders. International patent application WO 2006/040103 (PTL 2) purportedly describes methods and pharmaceutical compositions for treatment and/or prophylaxis of respiratory diseases or disorders. Recently, International patent application WO 2014/025651 (PTL 3) from Amgen Inc. purportedly describes chroman compounds and derivatives as TRPM8 inhibitors for the treatment of migraines and neuropathic pain.

Recently, WO 2015/108136 (PTL 4) and US2015/0158875 (PTL 5) disclose TRPM8 receptor antagonists. Each chemical structure is alpha-substituted glycinamide derivative and a substituted aza-bicyclic imidazole derivative, respectively, which is quite different from an imidazolinone derivative of the present invention.

An imidazolinone derivative of the present invention which has TRPM8 receptor antagonist activity has never been known.

### CITATION LIST

### Patent Literature

{PTL 1} WO 2006/040136
{PTL 2} WO 2006/040103
{PTL 3} WO 2014/025651
{PL 4} WO 2015/108136
{PTL 5} US2015/0158875

### Non Patent Literature

{NPL 1} McKemy, D. D., et al., Nature 416, 52-58, 2002
{NPL 2} Peier, A. M., Cell 108, Issue 5, 705-715, 2002
{NPL 3} Abe, J., et al., Neurosci Lett, 397(1-2), 140-144, 2006
{NPL 4} Premkumar, L. S. et atl, J. Neurosci, 25(49), 11322-11329, 2005
{NPL 5} Kobeyashi, K., et al., J Comp Neurol, 493(4), 596-606, 2005
{NPL 6} Roza, C., et al., Pain, 120(1-2), 24-35, 2006
{NPL 7} Xing, H., et al., J Neurophysiol, 95(2), 1221-1230, 2006
{NPL 8} European Journal of Pharmacology, Volume 716, Issues 1-3, 61-76, 2013
{NPL 9} PAIN, Volume 152, Issue 10, 2211-2223, 2011
{NPL 10} Gauchan, P., et al., Neurosci Lett, 458, 93-95, 2009
{NPL 11} Sachin, S. Chaudhari, et al., Bioorg. Med. Chem. 21, 6542-6553, 2013

### SUMMARY OF INVENTION

### Technical Problem

There is a need in the art for TRPM8 antagonists that can be used to treat a disease, syndrome, or condition in a mammal wherein the disease, syndrome, or condition is affected by the modulation of TRPM8 receptors, such as wherein the condition or disorder is one or more of inflammatory, pain and urological diseases or disorders, including chronic pain; neuropathic pain including cold allodynia and diabetic neuropathy; postoperative pain; osteoarthritis; rheumatoid arthritic pain; cancer pain; neuralgia; neuropathies; algesia; dentin hypersensitivity; nerve injury; migraine; cluster and tension headache; ischaemia; irritable bowel syndrome; Raynaud's syndrome; nenrodegeneration; fibromyalgia; stroke; itch; psychiatric disorders including anxiety and depression; inflammatory disorders including asthma, chronic obstructive pulmonary, airways disease including COPD (chronic obstructive pulmonary disease), pulmonary hypertension; anxiety including other stress-related disorders; and urological diseases or disorders including detrusor overactivity or overactive bladder, urinary incontinence, neurogenic detrusor overactivity or detrusor hyperreflexia, idiopathic detrusor overactivity or detrusor instability, benign prostatic hyperplasia, and lower urinary tract symptoms; and combinations thereof.

TRPM8 antagonists should be well absorbed from the GI tract and should be metabolically stable and possess favorable pharmacokinetic properties. They should be non-toxic. Furthermore, the ideal drug candidate would exist in a physical form that is stable, non-hygroscopic and easily formulated. In particular, it has been desired that compounds would have to bind potently to the TRPM8 receptor and show functional activity as antagonists. The present invention provides novel compounds which have excellent TRPM8 antagonistic activities.

### Solution to Problem

With respect to other compounds disclosed in the art, the compounds of the present invention may show less toxicity, good absorption and distribution, good solubility, less plasma protein binding, less drug-drug interaction, good metabolic stability, reduced inhibitory activity at HERG (Human ether-ago-go-related gene) channel, and/or reduced QT prolongation.

The present invention provides:

[1] a compound of the following formula (I)

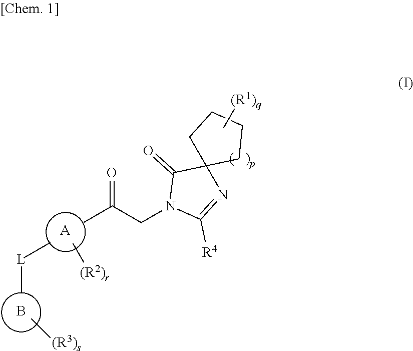

wherein
A is aryl and heteroaryl;
B is aryl and heteroaryl;
L is independently selected from the group consisting of a chemical bond, oxygen, sulfur, —NR⁵—, —(CR^{A}R^{B})ₜ—, —O(CR^{A}R^{B})ₜ—, —(CR^{A}R^{B})ₜO—, —N(R⁵)(CR^{A}R^{B})ₜ—, —(CR^{A}R^{B})ₜN(R⁵)—, —N(R⁵)(CR^{A}R^{B})ₜO—, and —O(CR^{A}R^{B})ₜN(R⁵)—R^{A} and R^{B} are independently selected from the group consisting of (1) hydrogen, (2) halogen, (3) (C₁-C₁₀)alkyl, (4) (C₁-C₁₀)cycloalkyl and (5) (C₁-C₁₀)haloalkyl; or R^{A} and R^{B} may form a 3 to 8 membered ring which may contain one or more heteroatoms independently selected from oxygen, sulfur and nitrogen; and said ring is optionally substituted with 1 to 6 substituents independently selected from (1) hydrogen, (2) halogen, (3) hydroxy, (4) (C₁-C₁₀)alkyl, (5) (C₁-C₁₀)cycloalky, (6) (C₁-C₁₀)haloalkyl, (7) (C₁-C₁₀)alkoxy and (8) (C₁-C₁₀)haloalkoxy;
R¹ is independently selected from the group consisting of (1) hydrogen, (2) halogen, (3) amino, (4) cyano, (5) hydroxyl, (6) (C₁-C₁₀)alkyl, (7) (C₃-C₁₀)cycloalkyl, (8) (C₁-C₁₀)haloalkyl, (9) (C₃-C₁₀)alkoxy and (10) (C₁-C₁₀)haloalkoxy; two R¹ on the same carbon or the different carbons are possible to form a 3 to 8 membered ring which may contain an atom selected from oxygen, sulfur and nitrogen; and said ring is optionally substituted with 1 to 6 substituents independently selected from (1) hydrogen, (2) halogen, (3) hydroxy, (4) (C₁-C₁₀)alkyl, (5) (C₃-C₁₀)cycloalkyl, (6) (C₁-C₁₀)haloalkyl, (7) (C₁-C₁₀)alkoxy, and (8) (C₁-C₁₀)haloalkoxy;
R² is independently selected from the group consisting of (1) hydrogen, (2) halogen, (3) amino, (4) —NH(C₁-C₆)alkyl, (5) —N[(C₁-C₆)alkyl]₂ wherein the alkyl is same or different, (6) cyano, (7) hydroxyl, (8) nitro, (9) (C₁-C₆)alkylthio, (10) (C₁-C₁₀)alkyl, (11) (C₃-C₁₀)cycloalkyl, (12) (C₁-C₁₀)alkoxy, (13) (C₁-C₁₀)haloalkyl and (14) (C₁-C₁₀)haloalkoxy;
R³ is independently selected from the group consisting of (1) hydrogen, (2) halogen, (3) cyano, (4) nitro, (5) hydroxyl, (6) (C₁-C₆)alkylthio, (7) (C₁-C₆)alkylsulfinyl, (8) (C₁-C₆)alkylsulfonyl, (9) —NR⁶R⁷, (10) —C(═O)NR⁶R⁷, (11) tri(C₁-C₆)alkylsilyl, (12) (C₁-C₁₀)alkyl, (13) (C₃-C₁₀)cycloalkyl, (14) (C₁-C₆)alkoxy(C₀-C₆)alkyl, (15) (C₃-C₁₀)cycloalkoxy, (16) —C(═O)(C₁-C₆)alkyl, (17) —C(═O)O(C₁-C₆)alkyl and (18) —C(═O)OH; said (C₁-C₁₀)alkyl. (C₃-C₁₀)cycloalkyl, (C₁-C₆)alkoxy(C₀-C₆)alkyl and (C₃-C₁₀)cycloalkoxy are optionally substituted with 1 to 6 substituents independently selected from (1) hydrogen, (2) halogen, (3) hydroxyl, (4) cyano, (5) (C₃-C₁₀)cycloalkyl, (6) (C₁-C₁₀)haloalkyl, (7) (C₁-C₁₀)alkoxy, (8) (C₁-C₁₀)haloalkoxy and (9) —NR⁶R⁷;
wherein R⁶ and R⁷, together with nitrogen atom to which they are attached, may form a 3 to 10 membered ring which may contain an atom selected from oxygen, sulfur and nitrogen; and said ring is optionally substituted with 1 to 6 substituents independently selected from (1) hydrogen, (2) halogen, (3) hydroxyl, (4) (C₁-C₁₀)alkyl, (5) (C₁-C₁₀)cycloalkyl, (6) (C₁-C₁₀)haloalkyl, (7) (C₁-C₁₀)alkoxy and (8) (C₁-C₁₀)haloalkoxy;
R⁴ is independently selected from the group consisting of (1) hydrogen, (2) (C₁-C₁₀)alkyl (3) (C₁-C₁₀)cycloalkyl and (4) (C₁-C₁₀)haloalkyl;
R⁵, R⁶ and R⁷ are independently selected from the group consisting of (1) hydrogen, (2) (C₁-C₁₀)alkyl, (3) (C₃-C₁₀)cycloalkyl, (4) (C₁-C₁₀)haloalkyl, (5) hydroxyl(C₁-C₁₀)alkyl, (6) (C₁-C₁₀)alkoxy(C₁-C₁₀)alkyl, (7) H₂N—(C₁-C₁₀)alkyl, (8) [(C₁-C₁₀)alkyl]NH—(C₁-C₁₀)alkyl, (9) [(C₁-C₁₀)alkyl]₂N—(C₁-C₁₀)alkyl, (10) (C₁-C₁₀)alkylcarbonyl and (11) (C₁-C₁₀)alkylsulfonyl;

p is 1, 2, 3 or 4;

q is 1, 2, 3 or 4; when q is two or more than two, R¹ is same or different,

r is 1, 2, 3 or 4; when r is two or more than two, R² is same or different,

s is 1, 2, 3, 4, 5, 6 or 7; when s is two or more than two, R³ is same or different,

t is 1, 2 or 3; when t is two or more than two, R^{A} and R^{B} are same or different, or a pharmaceutically acceptable salt thereof or a prodrug thereof;

[2] the compound described in [1] wherein

A is 6 membered aryl or 5 to 6 membered heteroaryl or a pharmaceutically acceptable salt thereof or a prodrug thereof;

[3] the compound described in [1] or [2] wherein

A is independently selected from the group consisting of benzene, pyridine, pyridazine, pyrazine, pyrimidine, triazine, thiophene, furan, pyrrole, imidazole, pyrazole, thiazole, isothiazole, oxazole, isoxazole, and triazole, or a pharmaceutically acceptable salt thereof or a prodrug thereof;

[4] The compound as described in any one of [1] to [3] which is selected from:
8,8-difluoro-2-methyl-3-(2-oxo-2-(4-(pyridazin-3-yloxy)phenyl)ethyl)-1,3-diazaspiro[4.5]dec-1-en-4-one;
2-methyl-3-(2-(4-(2-methyl-1H-benzo[d]imidazol-1-yl)phenyl)-2-oxoethyl)-1,3-diaz aspiro[4.5]dec-1-en-4-one;
2-ethyl-8,8-difluoro-3-(2-oxo-2-(4-(pyridazin-3-yloxy)phenyl)ethyl)-1,3-diazaspiro[4.5]dec-1-en-4-one;
8,8-difluoro-2-methyl-3-(2-(4-(4-methylpyridazin-3-yl)phenyl)-2-oxoethyl)-1,3-diazaspiro[4,5]dec-1-en-4-one;
8,8-difluoro-2-methyl-3-(2-(4-(2-methyl-1H-benzo[d]imidazol-1-yl)phenyl)-2-oxoethyl)-1,3-diazaspiro[4.5]dec-1-en-4-one;
8,8-difluoro-2-methyl-3-(2-(4-(2-methyl-3H-imidazo[4,5-b]pyridin-3-yl)phenyl)-2-oxo ethyl)-1,3-diazaspiro[4.5]dec-1-en-4-one;
   or a pharmaceutically acceptable salt thereof or a prodrug thereof.

[5] a use of a compound described in any one of [1] to [4] or a pharmaceutically acceptable salt thereof or a prodrug thereof for the manufacture of a medicament for the treatment of a condition or disorder mediated by TRPM8 receptor antagonistic activity;

[6] the use as described in [5], wherein the condition or disorder is one or more of inflammatory, pain and urological diseases or disorders, including chronic pain; neuropathic pain including cold allodynia and diabetic neuropathy; postoperative pain; osteoarthritis; rheumatoid arthritic pain; cancer pain; neuralgia; neuropathies; algesia; dentin hypersensitivity; nerve injury; migraine; cluster and tension headache; ischaemia; irritable bowel syndrome; Raynaud's syndrome; neurodegeneration; fibromyalgia; stroke; itch; psychiatric disorders including anxiety and depression; inflammatory disorders including asthma, chronic obstructive pulmonary, airways disease including COPD, pulmonary hypertension; anxiety including other stress-related disorders; and urological diseases or disorders including detrusor overactivity or overactive bladder, urinary incontinence, neurogenic detrusor overactivity or detrusor hyperreflexia, idiopathic detrusor overactivity or detrusor instability, benign prostatic hyperplasia, and lower urinary tract symptoms; and combinations thereof;

[7] a method for the treatment of a condition or disorder mediated by TRPM8 receptor antagonistic activity in a mammalian subject, including a human, which comprises administering to a mammal in need of such treatment a therapeutically effective amount of a compound described in any one of [1] to [4] or a pharmaceutically acceptable salt thereof or a prodrug thereof;

[8] the method as described in [7], wherein the condition or disorder is one or more of inflammatory, pain and urological diseases or disorders, including chronic pain; neuropathic pain including cold allodynia and diabetic neuropathy; postoperative pain; osteoarthritis; rheumatoid arthritic pain; cancer pain; neuralgia; neuropathies; algesia; dentin hypersensitivity; nerve injury; migraine; cluster and tension headache; ischaemia; irritable bowel syndrome; Raynaud's syndrome; neurodegeneration; fibromyalgia; stroke; itch; psychiatric disorders including anxiety and depression; inflammatory disorders including asthma, chronic obstructive pulmonary, airways disease including COPD, pulmonary hypertension; anxiety including other stress-related disorders; and urological diseases or disorders including detrusor overactivity or overactive bladder, urinary incontinence, neurogenic detrusor overactivity or detrusor hyperreflexia, idiopathic detrusor overactivity or detrusor instability, benign prostatic hyperplasia, and lower urinary tract symptoms; and combinations thereof;

[9] a pharmaceutical composition comprising a compound or a pharmaceutically acceptable salt thereof or a prodrug thereof, as described in any one of [1] to [4], and a pharmaceutically acceptable carrier,

[10] the pharmaceutical composition as described in [9], further comprising another pharmacologically active agent;

[11] a compound described in any one of [1] to [4] or a pharmaceutically acceptable salt thereof or a prodrug thereof for use in the treatment of a condition or disorder mediated by TRPM8 receptor antagonistic activity; and

[12] a process for preparing a pharmaceutical composition, wherein the process comprises mixing a compound described in any one of [1] to [4] or a pharmaceutically acceptable salt thereof or a prodrug thereof and a pharmaceutically acceptable carrier or excipient.

Examples of conditions or disorders mediated by TRPM8 receptor activity include, but are not limited to, TRPM8 related diseases.

### Advantageous Effects of Invention

The compounds of the present invention show the TRPM8 receptor antagonistic activity. The compounds of the present invention may show less toxicity, good absorption, distribution, good solubility, less protein binding affinity other than TRPM8 receptor, less drug-drug interaction, and good metabolic stability.

### DESCRIPTION OF EMBODIMENTS

As used herein, the term “alkyl” as a group or part of a group e.g. alkoxy or hydroxyalkyl refers to a straight or branched alkyl group in all isomeric forms. The term “C₁-C₄ alkyl” refers to an alkyl group, as defined above, containing at least 1 and at most 4 carbon atoms. Examples of such alkyl groups include methyl, ethyl, propyl, isopropyl, n-butyl, iso-butyl, sec-butyl, or tert-butyl. Examples of such alkoxy groups include methoxy, ethoxy, propoxy, iso-propoxy, butoxy, iso-butoxy, sec-butoxy and tert-butoxy.

The term “cycloalkyl”, as used herein, means a mono- or bicyclic ring, but not limited to, cyclopropyl, cyclobutyl, cyclopentyl, cyclohexyl, cycloheptyl, norbornyl, adamantyl groups and the like.

Then cyclopropylmethyl and cyclopentylmethyl are as follows:

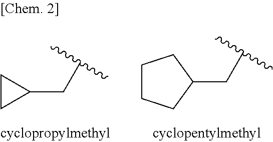 [SMILES: *CC1CC1 ; *CC1CCCC1]

The term “halogen” refers to fluorine (F), chlorine (Cl), bromine (Br), or iodine (I) and the term “halo” refers to the halogen: fluoro (—F), chloro (—Cl), bromo (—Br) and iodo (—I).

The term “haloalkyl”, as used herein, means an alkyl radical which is substituted by halogen atom(s) as defined above including, but not limited to, fluoromethyl, difluoromethyl, trifluoromethyl, 2-fluoroethyl, 2,2-difluoroethyl, 2,2,2-trifluoroethyl, 2,2,2-trichloroethyl, 3-fluoropropyl, 4-fluorobutyl, chloromethyl, trichloromethyl, iodomethyl, bromomethyl groups and the like.

The term “haloalkoxy”, as used herein, means haloalkyl-O—, including, but not limited to, fluoromethoxy, difluoromethoxy, trifluoromethoxy, 2-fluoroethoxy, 2,2-difluoroethoxy, 2,2,2-trifluroethoxy, 2,2,2-trichloroethoxy, 3-fluoropropoxy, 4-fluorobutoxy, chloromethoxy, trichloromethoxy, iodomethoxy, bromomethoxy groups and the like.

The term “alkoxy”, as used herein, means an O-alkyl group wherein “alkyl” is defined above.

The term “heterocyclyl”, as used herein, means a saturated 3- to 16-membered ring which comprises one or more heteroatoms selected from nitrogen, oxygen and sulfur. For purposes of this invention, the heterocyclyl may be a monocyclic, bicyclic or tricyclic ring system, which may include fused, bridged or spiro ring systems. Examples of such heterocyclyl groups include azetidinyl, 1,4-dioxanyl, pyrrolidinyl, piperidinyl, piperazinyl, morpholinyl, tetrahydrofuranyl, thiomorpholinyl, tetrahydrothienyl, 2-oxo-pyrrolidinyl, 2-oxo-piperidinyl, 2-oxo-imidazolidinyl, 2-oxo-oxazolidinyl, quinuclidinyl, azabicyclo[3.2.1]octyl, 2-oxa-6-azaspiro[3.4]octyl and N-oxides thereof and S-oxides thereof.

The term “aryl”, as used herein, means unsaturated and partially saturated 6- to 15-membered ring which consists of carbon atoms;

Examples of such unsaturated aryl include, but are not limited to, phenyl, naphthyl, indanyl, indenyl, 1,2,3,4-tetrahydronaphthyl, and 1,2-dihydronaphthyl.

The term “heteroaryl” as used herein, means 5- to 15-membered ring, preferably 6- to 15-membered ring, in which an aromatic heteroatom containing ring is fused to a non-aromatic ring, such as heterocyclyl ring or cycloalkyl ring, and also means 5- to 15-membered ring, preferably 6- to 15-membered ring, in which an aryl ring is fused to a non-aromatic heteroatom containing ring, such as heterocyclyl ring.

Namely, the term “heteroaryl” as used herein, means the following:
1) unsaturated and partially saturated 5- to 15-membered ring, preferably 6- to 15-membered ring, which consists of carbon atoms and from one to five heteroatoms selected from nitrogen, phosphorus, oxygen and sulfur;
2) unsaturated and partially saturated 5- to 15-membered ring, preferably 6- to 15-membered ring, in which a non-aromatic ring, such as heterocyclyl ring or cycloalkyl ring, is fused to a heteroaryl defined above; and
3) unsaturated and partially saturated 5- to 15-membered ring, preferably 6- to 15-membered ring, in which an aryl ring is fused to a heterocyclyl ring.
Examples of such heteroaryl include, but are not limited to, thiophenyl, thiazolyl, isoxazolyl, pyrazolyl, tetrazolyl, furanyl, pyrrolyl, imidazolyl, oxazolyl, isothiazolyl, triazolyl, thiadiazolyl, pyridyl, pyrimidyl, pyridazinyl, pyrazinyl, triazinyl, benzofuranyl, benzothiophenyl, benzotriazolyl, indolyl, indazolyl, benzoimidazolyl, pyrrolopyridyl, pyrrolopyrimidinyl, pyrazolopyridyl, pyrazolopyrimidinyl, imidazopyridinyl, furopyridyl, benzoisoxazolyl, imidazopyrazinyl, imidazopyridazinyl, inidazopyrimidinyl, quinolyl, isoquinolyl, quinazolinyl, phthalazinyl, quinoxalinyl, naphthyridinyl, pyridopyrimidinyl, and N-oxides thereof and S-oxides thereof.

Examples of such heteroaryl also include the heteroaryl ring radical consisting of the following rings.

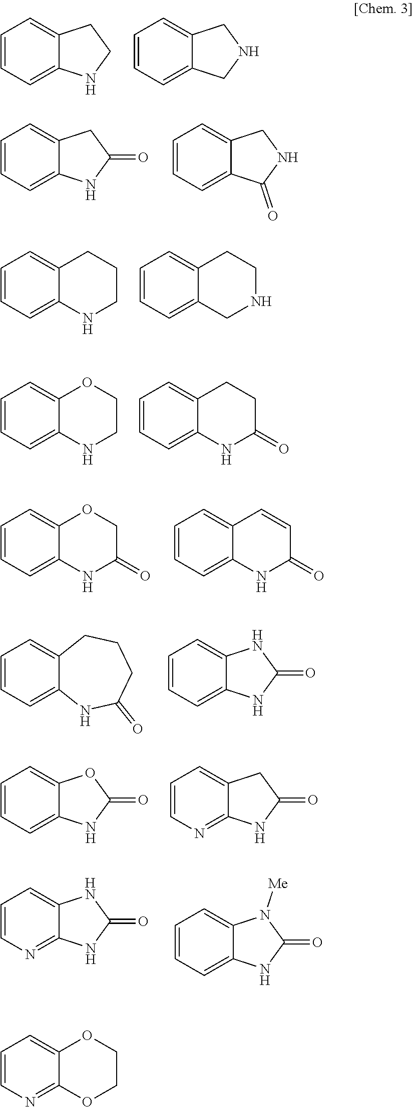

The term “C₀”, as used herein, means direct bond.

The substituents on the ring of the compound of the present invention may exist on the any atoms if it is chemically allowed.

The term “protecting group”, as used herein, means a hydroxy or amino protecting group which is selected from typical hydroxy or amino protecting groups described in Protective Groups in Organic Synthesis Forth Edition edited by T. W. Greene et al. (John Wiley & Sons, 2006);

The terms “treating” and “treatment”, as used herein, refer to curative, palliative and prophylactic treatment, including reversing, alleviating, inhibiting the progress of, or preventing the disorder or condition to which such term applies, or one or more symptoms of such disorder or condition.

As used herein, the article “a” or “an” refers to both the singular and plural form of the object to which it refers unless indicated otherwise.

The symbol letter is written the corresponding English word in the present specification. For example, the symbols α, β, and δ are written alpha, beta, and delta, respectively.

Included within the scope of the “compounds of the invention” are all salts, solvates, hydrates, complexes, polymorphs, prodrugs, radiolabeled derivatives, stereoisomers and optical isomers of the compounds of formula (I).

The compounds of formula (I) can form acid addition salts thereof. It would be appreciated that for use in medicine the salts of the compounds of formula (I) should be pharmaceutically acceptable. Suitable pharmaceutically acceptable salts would be apparent to those skilled in the art and include those described in J. Pharm. Sci, 66, 1-19, 1977, such as acid addition salts formed with inorganic acids e.g. hydrochloric, hydrobromic, sulfuric, nitric or phosphoric acid; and organic acids e.g. succinic, maleic, formic, acetic, trifluoroacetic, propionic, fumaric, citric, tartaric, benzoic, p-toluenesulfonic, methanesulfonic or naphthalenesulfonic acid. Certain of the compounds of formula (I) may form acid addition salts with one or more equivalents of the acid. The present invention includes within its scope all possible stoichiometric and non-stoichiometric forms. In addition, certain compounds containing an acidic function such as a carboxy can be isolated in the form of their inorganic salt in which the counter ion can be selected from sodium, potassium, lithium, calcium, magnesium and the like, as well as from organic bases.

Also within the scope of the invention are so-called “prodrugs” of the compounds of formula (I). Thus certain derivatives of compounds of formula (I) which may have little or no pharmacological activity themselves, when administered into or onto the body, can be converted into compounds of formula (I) having the desired activity, for example, by hydrolytic cleavage. Such derivatives are referred to as “prodrugs”. Further information on the use of prodrugs may be found in Pro-drugs as Novel Delivery Systems, Vol. 14, ACS Symposium Series (T Higuchi and V Stella) and Bioreversible Carriers in Drug Design, Pergamon Press, 1987 (ed. E B Roche, American Pharmaceutical Association).

Prodrugs in accordance with the invention, for example, can be produced by replacing appropriate functionalities present in the compounds of formula (I) with certain moieties known to those skilled in the art as ‘pro-moieties’ as described, for example, in Design of Prodrugs by H Bundgaard (Elsevier, 1985). Some examples of prodrugs in accordance with the invention include:

(i) where the compound of formula (I) contains an alcohol functionality (—OH), compounds wherein the hydroxy group is replaced with a moiety convertible in vivo into the hydroxy group. Said moiety convertible in vivo into the hydroxy group means a moiety transformable in vivo into a hydroxyl group by e.g. hydrolysis and/or by an enzyme, e.g. an esterase. Examples of said moiety include, but are not limited to, ester and ether groups which may be hydrolyzed easily in vivo. Preferred are the moieties replaced the hydrogen of hydroxy group with acyloxyalkyl, 1-(alkoxycarbonyloxy)alkyl, phthalidyl and acyloxyalkyloxycarbonyl such as pivaloyloxymethyloxycarbonyl.

(ii) where the compound of the formula (I) contains an amino group, an amide derivative prepared by reacting with a suitable acid halide or a suitable acid anhydride is exemplified as a prodrug. A particularly preferred amide derivative as a prodrug is —NHCO(CH₂)₂OCH₃, —NHCOCH(NH₂)CH₃ or the like.

Further examples of replacement groups in accordance with the foregoing examples and examples of other prodrug types may be found in the aforementioned references.

The compounds of formula (I), salts thereof and prodrugs thereof may be prepared in crystalline or non-crystalline form and, if crystalline, may optionally be hydrated or solvated. This invention includes within its scope stoichiometric hydrates or solvates as well as compounds containing variable amounts of water and/or solvent.

Salts and solvates having non-pharmaceutically acceptable counter-ions or associated solvents are within the scope of the present invention, for example, for use as intermediates in the preparation of other compounds of formula (I) and their pharmaceutically acceptable salts.

Additionally, the compounds of formula (I) may be administered as prodrugs. As used herein, a “prodrug” of a compound of formula (I) is a functional derivative of the compound which, upon administration to a patient, eventually liberates the compound of formula (I) in vivo. Administration of a compound of formula (I) as a prodrug may enable the skilled artisan to do one or more of the followings: (a) modify the onset of action of the compound in vivo; (b) modify the duration of action of the compound in vivo; (c) modify the transportation or distribution of the compound in vivo; (d) modify the solubility of the compound in vivo; and (e) overcome a side effect or other difficulty encountered with the compound. Typical functional derivatives used to prepare prodrugs include modifications of the compound that are chemically or enzymatically cleaved in vivo. Such modifications, which include the preparation of phosphates, amides, esters, thioesters, carbonates, and carbamates, are well known to those skilled in the art.

In certain of the compounds of formula (I), there may be some chiral carbon atoms. In such cases, compounds of formula (I) exist as stereoisomers. The invention extends to all optical isomers such as stereoisomeric forms of the compounds of formula (I) including enantiomers, diastereoisomers and mixtures thereof, such as racemates. The different stereoisomeric forms may be separated or resolved one from the other by conventional methods or any given isomer may be obtained by conventional stereoselective or asymmetric syntheses.

Certain of the compounds herein can exist in various tautomeric forms and it is to be understood that the invention encompasses all such tautomeric forms.

The invention also includes isotopically-labeled compounds, which are identical to those described herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine, iodine, and chlorine, such as ³H, ¹¹C, ¹⁴C, ¹⁸F, ¹²³I and ¹²⁵I. Compounds of the invention that contain the aforementioned isotopes and/or other isotopes of other atoms are within the scope of the present invention. Isotopically-labeled compounds of the present invention, for example those into which radioactive isotopes such as ³H, ¹⁴C are incorporated, am useful in drug and/or substrate tissue distribution assays. Tritiated, i.e., ³H, and carbon-14, i.e., ¹⁴C, isotopes are particularly preferred for their ease of preparation and detectability. ¹¹C and ¹⁸F isotopes are particularly useful in PET (positron emission tomography), and ¹²⁵I isotopes are particularly useful in SPECT (single photon emission computerized tomography), all useful in brain imaging. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the invention can be generally prepared by carrying out the procedures disclosed in the Schemes and/or in the Examples below, then substituting a readily available isotopically labeled reagent for a non-isotopically labeled reagent.

The potencies and efficacies of the compounds of this invention for TRPM8 can be determined by reporter assay performed on the human cloned receptor as described herein. Compounds of formula (I) have demonstrated antagonistic activity at the TRPM8 receptor, using the functional assay described herein.

Compounds of formula (I) and pharmaceutically acceptable salts thereof are therefore of use in the treatment of conditions or disorders which are mediated via the TRPM8 receptor. In particular the compounds of formula (I) and pharmaceutically acceptable salts thereof are of use in the treatment of a wide range of diseases, syndromes, and disorders, in particular for the treatment of inflammatory, pain and urological diseases or disorders, such as wherein the condition or disorder is one or more of inflammatory, pain and urological diseases or disorders, including chronic pain; neuropathic pain including cold allodynia and diabetic neuropathy; postoperative pain; osteoarthritis; rheumatoid arthritic pain; cancer pain; neuralgia; neuropathies; algesia; dentin hypersensitivity; nerve injury; migraine; cluster and tension headache; ischaemia; irritable bowel syndrome; Raynaud's syndrome; neurodegeneration; fibromyalgia; stroke; itch; psychiatric disorders including anxiety and depression; inflammatory disorders including asthma, chronic obstructive pulmonary, airways disease including COPD, pulmonary hypertension; anxiety including other stress-related disorders; and urological diseases or disorders including detrusor overactivity or overactive bladder, urinary incontinence, neurogenic detrusor overactivity or detrusor hyperreflexia, idiopathic detrusor overactivity or detrusor instability, benign prostatic hyperplasia, and lower urinary tract symptoms; and combinations thereof.

Activities of the compound (I) for each disease, syndrome, and disorder described above can be confirmed in the suitable model known to skilled in the art. For example, activities of compounds of formula (I) for neuropathic pain have been confirmed in chronic constriction injury (CCI)-induced model, such as cold allodynia and static allodynia model.

It is to be understood that “treatment” as used herein includes prophylaxis as well as alleviation of established symptoms as described above.

A pharmaceutical composition of the invention, which may be prepared by admixture, suitably at ambient temperature and atmospheric pressure, is usually adapted for oral, parenteral or rectal administration and, as such, may be in the form of tablets, capsules, oral liquid preparations, powders, granules, lozenges, reconstitutable powders, injectable or infusible solutions or suspensions or suppositories. Orally administered compositions are generally preferred. Tablets and capsules for oral administration may be in unit dose form, and may contain conventional excipients, such as binding agents (e.g. pregelatinised maize starch, polyvinylpyrrolidone or hydroxypropyl methylcellulose); fillers (e.g. lactose, microcrystalline cellulose or calcium hydrogen phosphate); tableting lubricants (e.g. magnesium stearate, talc or silica); disintegrants (e.g. potato starch or sodium starch glycolate); and acceptable wetting agents (e.g. sodium lauryl sulphate). The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents (e.g. sorbitol syrup, cellulose derivatives or hydrogenated edible fats), emulsifying agents (e.g. lecithin or acacia), non-aqueous vehicles (which may include edible oils e.g. almond oil, oily esters, ethyl alcohol or fractionated vegetable oils), preservatives (e.g. methyl or propyl-p-hydroxybenzoates or sorbic acid), and, if desired, conventional flavourings or colorants, buffer salts and sweetening agents as appropriate. Preparations for oral administration may be suitably formulated to give controlled release of the active compound or pharmaceutically acceptable salt thereof.

For parenteral administration, fluid unit dosage forms are prepared utilising a compound of formula (I) or pharmaceutically acceptable salt thereof and a sterile vehicle. Formulations for injection may be presented in unit dosage form e.g. in ampoules or in multi-dose, utilising a compound of formula (I) or pharmaceutically acceptable salt thereof and a sterile vehicle, optionally with an added preservative. The compositions may take such forms as suspensions, solutions or emulsions in oily or aqueous vehicles, and may contain formulatory agents such as suspending, stabilising and/or dispersing agents. Alternatively, the active ingredient may be in powder form for constitution with a suitable vehicle, e.g. sterile pyrogen-free water, before use. The compound, depending on the vehicle and concentration used, can be either suspended or dissolved in the vehicle. In preparing solutions, the compound can be dissolved for injection and filter sterilised before filling into a suitable vial or ampoule and sealing. Advantageously, adjuvants such as a local anaesthetic, preservatives and buffering agents are dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilisation cannot be accomplished by filtration. The compound can be sterilised by exposure to ethylene oxide before suspension in a sterile vehicle. Advantageously, a surfactant or wetting agent is included in the composition to facilitate uniform distribution of the compound.

Lotions may be formulated with an aqueous or oily base and in general will also contain one or more emulsifying agents, stabilising agents, dispersing agents, suspending agents, thickening agents, or colouring agents. Drops may be formulated with an aqueous or non-aqueous base also comprising one or more dispersing agents, stabilising agents, solubilising agents or suspending agents. They may also contain a preservative.

The compounds of formula (I) or pharmaceutically acceptable salts thereof may also be formulated in rectal compositions such as suppositories or retention enemas, e.g. containing conventional suppository bases such as cocoa butter or other glycerides.

The compounds of formula (I) or pharmaceutically acceptable salts may also be formulated as depot preparations. Such long acting formulations may be administered by implantation (for example subcutaneously or intramuscularly) or by intramuscular injection. Thus, for example, the compounds of formula (I) or pharmaceutically acceptable salts may be formulated with suitable polymeric or hydrophobic materials (for example as an emulsion in an acceptable oil) or ion exchange resins, or as sparingly soluble derivatives, for example, as a sparingly soluble salt.

For intranasal administration, the compounds formula (I) or pharmaceutically acceptable salts thereof may be formulated as solutions for administration via a suitable metered or unitary dose device or alternatively as a powder mix with a suitable carrier for administration using a suitable delivery device. Thus the compounds of formula (I) or pharmaceutically acceptable salts thereof may be formulated for oral, buccal, parenteral, topical (including ophthalmic and nasal), depot or rectal administration or in a form suitable for administration by inhalation or insufflation (through either mouth or nose). The compounds of formula (I) and pharmaceutically acceptable salts thereof may be formulated for topical administration in the form of ointments, creams, gels, lotions, pessaries, aerosols or drops (e.g. eye, ear or nose drops). Ointments and creams may for example, be formulated with an aqueous or oily base with the addition of suitable thickening and/or gelling agents. Ointments for administration to the eye may be manufactured in a sterile manner using sterilized components.

A TRPM8 antagonist may be usefully combined with another pharmacologically active compound, or two or more other pharmacologically active compounds, particularly in the treatment of inflammatory, pain and urological diseases or disorders. For example, a TRPM8 antagonist, particularly a compound of formula (I), or a pharmaceutically acceptable salt or solvate thereof, as defined above, may be administered simultaneously, sequentially or separately in combination with one or more agents selected from:
an opioid analgesic, e.g. morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine or pentazocine;
a nonsteroidal antiinflammatory drug (NSAID), e.g. aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, meloxicam, nabumetone, naproxen, nimesulide, nitroflurbiprofen, olsalazine, oxaprozin, phenylbutazone, piroxicam, sulfasalazine, sulindac, tolmetin or zomepirac:
a barbiturate sedative, e.g. amobarbital, aprobarbital, butabarbital, butabital, mephobarbital, metharbital, methohexital, pentobarbital, phenobartital, secobarbital, talbutal, thiamylal or thiopental;
a benzodiazepine having a sedative action, e.g. chlordiazepoxide, clorazepate, diazepam, flurazepam, lorazepam, oxazepam, temazepam or triazolam;
an H1 antagonist having a sedative action, e.g. diphenhydramine, pyrilamine, promethazine, chlorpheniramine or chlorcyclizine;
a sedative such as glutethimide, meprobamate, methaqualone or dichloralphenazone;
a skeletal muscle relaxant, e.g. baclofen, carisoprodol, chlorzoxazone, cyclobenzaprine, methocarbamol or orphenadrine;
an NMDA receptor antagonist, e.g. dextromethorphan ((+)-3-methoxy-N-methylmorphinan) or its metabolite dextrorphan ((+)-3-hydroxy-N-methylmorphinan), ketamine, memantine, pyrroloquinoline quinine, cis-4-(phosphonomethyl)-2-piperidinecarboxylic acid, budipine, EN-3231 (MorphiDex(registered trademark), a combination formulation of morphine and dextromethorphan), topiramate, neramexane or perzinfotel including an NR2B antagonist, e.g. ifenprodil, traxoprodil or (−)-(R)-6-{2-[4-(3-fluorophenyl)-4-hydroxy-1-piperidinyl]-1-hydroxyethyl}-3,4-dihydro-2(1H)-quinolinone;
an alpha-adrenergic, e.g. doxazosin, tamsulosin, clonidine, guanfacine, dexmedetomidine, modafinil, or 4-amino-6,7-dimethoxy-2-(5-methanesulfonamido-1,2,3,4-tetrahydroisoquinol-2-yl)-5-(2-pyridyl)quinazoline;
a tricyclic antidepressant, e.g. desipramine, imipramine, amitriptyline or nortriptyline;
an anticonvulsant, e.g. carbamazepine, lamotrigine, topiramate or valproate;
a tachykinin (NK) antagonist, particularly an NK-3, NK-2 or NK-1 antagonist, e.g. (alphaR,9R)-7-[3,5-bis(trifluoromethyl)benzyl]-8,9,10,11-tetrahydro-9-methyl-5-(4-methylphenyl)-7H-[1,4]diazocino[2,1-g][1,7]-naphthyridine-6,13-dione (TAK-637), 5-[[(2R,3S)-2-[(R)-1-[3,5-bis(trifluoromethyl)phenyl]ethoxy-3-(4-fluorophenyl)-4-morpholinyl]methyl]-1,2-dihydro-3H-1,2,4-triazol-3-one (MK-869, aprepitant), lanepitant, dapitant or 3-[[2-methoxy-5-(trifluoromethoxy)phenyl]methylamino]-2-phenylpiperidine (2S,3S);
a muscarinic antagonist, e.g. oxybutynin, tolterodine, propiverine, trospium chloride, darifenacin, solifenacin, temiverine and ipratropium;
a COX-2 selective inhibitor, e.g. celecoxib, rofecoxib, parecoxib, valdecoxib, deracoxib, etoricoxib, or lumiracoxib;
a coal-tar analgesic, in particular paracetamol;
a neuroleptic such as droperidol, chlorpromazine, haloperidol, perphenazine, thioridazine, mesoridazine, trifluoperazine, fluphenazine, clozapine, olanzapine, risperidone, ziprasidone, quetiapine, sertindole, aripiprazole, sonepiprazole, blonanserin, iloperidone, perospirone, raclopride, zotepine, bifeprunox, asenapine, lurasidone, amisuipride, balaperidone, palindore, eplivanserin, osanetant, rimonabant, meclinertant, Miraxion (registered trademark) or sarizotan;
a vanilloid receptor agonist (e.g. resiniferatoxin) or antagonist (e.g. capsazepine);
a transient receptor potential cation channel subtype (V1, V2, V3, V4, M8, M2, A1) agonist or antagonist;
a beta-adrenergic such as propranolol;
a local anaesthetic such as mexiletine;
a corticosteroid such as dexamethasone;
a 5-HT receptor agonist or antagonist, particularly a 5-HT1B/1D agonist such as eletriptan, sumatriptan, naratriptan, zolmitriptan or rizatiptan;
a 5-HT2A receptor antagonist such as R(+)-alpha-(2,3-dimethoxy-phenyl)-1-[2-(4-fluorophenylethyl)]-4-pipeuidinemethanol (MDL-100907);
a cholinergic (nicotinic) analgesic, such as ispronicline (TC-1734), (E)-N-methyl-4(3-pyridinyl)-3-buten-1-amine (RJR-2403), (R)-5-((2-azetidinylmethoxy)-2-chloropyridine (ABT-594) or nicotine;
Tramadol (registered trademark);
a PDEV inhibitor, such as
5-[2-ethoxy-5-{(4-methylpiperazin-1-yl)sulfonyl}phenyl]-1-methyl-3-propyl-1H-pyrazolo[4,3-d]pyrimidin-7(6H)-one (sildenafil),
(6R,2aR)-2,3,6,7,12,12a-hexahydro-2-methyl-6-(3,4-methylenedioxyphenyl)pyrazino[2′,1′:6,1]pyrido[3,4-b]indole-1,4-dione (IC-351 or tadalafil),
2-[2-ethoxy-5-{((4-ethylpiperazin-1-yl)sulfonyl}phenyl]-5-methyl-7-propylimidazo[5,1-f][1,2,4]triazin-4(3H)-one (vardenafil),
5-(5-acetyl-2-butoxy-3-pyridinyl)-3-ethyl-2-(l-ethyl-3-azetidinyl)-2,6-dihydro-7H-pyrazolo[4,3-d]pyrimidin-7-one,
5-(5-acetyl-2-propoxy-3-pyridinyl-3-ethyl-2-(1-isopropyl-3-azetidinyl)-2,6-dihydro-7H-pyrazolo[4,3-d]pyrimidin-7-one,
5-[2-ethoxy-5-{(4-ethylpiperazin-1-yl)sulfonyl}pyridin-3-yl]-3-ethyl-2-(2-methoxyethyl)-2H-pyrazolo[4,3-d]pyrimidin-7(6H1)-one,
4-[(3-chloro-4-methoxybenzyl)amino]-2-[(2S)-2-(hydroxymethyl)pyridin-1-yl]-N-(pyridin-2-yl)methyl)pyrimidine-5-carboxamide,
3-(1-methyl-7-oxo-3-propyl-6,7-dihydro-1H-pyrazolo[4,3-d]pyrimidin-5-yl)-N-[2-(1-methylpyrrolidin-2-yl)ethyl]-4-propoxybenzenesulfonamide;
   an alpha-2-delta ligand such as gabapentin, pregabalin, 3-methylgabapentin, (3-(aminomethyl)-bicyclo[3.2.0]hept 3-yl)acetic acid, (3S,5R)-3-(aminomethyl)-5-methylheptanoic acid, (3S,5R)-3-amino-5 methylheptanoic acid, (3S,5R)-3-amino-5-methyloctanoic acid, (2S,4S)-4-(3-chlorophenoxy)proline, (2S,4S)-4-(3-fluorobenzyl)proline, [(1R,5R,6S)-6-(aminomethyl)bicyclo[3.2.0]hept-6-yl]acetic acid, 3-((1-(aminomethyl)cyclohexyl)methyl)-4H-[1,2,4]oxadiazol-5-one, C[1-((1H-tetrazol-5-yl)methyl)cycloheptyl]methylamine. (3S,4S)-(1-(aminomethyl)-3,4-dimethylcyclopentyl)acetic acid, (3S,5R)-3-(aminomethyl)-5-methyloctanoic acid, (3S,5R)-3-amino-5-methylnonanoic acid, (3S,5R)-3-amino-5-methyloctanoic acid, (3R,4R,5R)-3-amino-4,5-dimethylheptanoic acid, and (3R,4R,5R)-3-amino-4,5-dimethyloctanoic acid;
   a cannabinoid;
   a metabotropic glutamate subtype 1 receptor (mGluR1) antagonist;
   a serotonin reuptake inhibitor such as sertraline, seitaline metabolite desmethlylsertraline, fluoxetine, norfluoxetine (fluoxetine desmethyl metabolite), fluvoxamine paroxetine, citalopram, citalopram metabolite desmethylcitalopram, escitalopramn, d,l-fenfluramine, femoxetine, ifoxetine, cyanodothiepin, litoxetine, dapoxetine, nefazodone, cericlamine and trazodone;
   a noradrenaline (norepinephrine) reuptake inhibitor, such as mnaprotiline, lofepramine, mirtazapine, oxaprotiline, fezolamine, tomoxetine, mianserin, bupropion, bupropion metabolite hydroxybupropion, nomifensine and viloxazine (Vivalan (registered trademark)), especially a selective noradrenaline reuptake inhibitor such as reboxetine, in particular (S,S)-reboxetine;
   a dual serotonin-noradrenatine reuptake inhibitor, such as venlafaxine, venlafaxine metabolite O-desmethylvenlafaxine, clomipramine, clomipramine metabolite desmethylclomipramine, duloxetine, milnacipran and imipramine;
   an inducible nitric oxide synthase (iNOS) inhibitor such as S[2-[(1-iminoethyl)amino]ethyl]-L-homocystine, S[2-[(1-iminoethyl)amino]ethyl]-4,4-dioxo-L-cysteine, S[2-[(1-iminoethyl)amino]ethyl]-2-methyl-L-cysteine, (2S,5Z)-2-amino-2-methyl-7-[(1-iminoethyl)amino]-5-heptenoic acid, 2-[[(1R,3S)-3-amino-4-hydroxy-1-(5-thiazolyl)butyl]thio]-5-chloro-3-pyridinecmrbonitrile; 2-[[(l R,3S)-3-amino-4-hydroxy-1-(5-thiazolyl)butyl]thio]-4-chlorobenzonitrile, (2S,4R)-2-amino-4-[[2-chloro-5-trifluoromethyl)phenyl]thio]-5-thiazolebutanol, 2-[[(1R,3S)-3-amino-4-hydroxy-1-(5-thiazolyl)butyl]thio]-6-(trifluoromethyl)-3-pyridinecarbonitrile, 2-[[(1R,3S)-3-amino 4-hydroxy-1-(5-thiazolyl)butyl]thio]-5-chlorobenzonitrile, N-[4-[2-(3-chlorobenzylamino)ethyl]phenyl]thiophene-2-carboxamidine, or guanidinoethyldisulfide;
   an acetylcholinesterase inhibitor such as donepezil;
   a prostaglandin E2 subtype 4 (EN) antagonist such as N[({2-[4-(2-ethyl-4,6-dimethyl-1H-imidazo[4,5-c]pyridin-1-yl)phenyl]ethyl}amino)-carbonyl]-4-methylbenzenesulfonamide or 4-[(1S)-1-({[5-chloro-2-(3-fluorophenoxy)pyridin-3-yl]carbonyl}amino)ethyl]benzoic acid;
   a leukotriene B4 antagonist; such as 1-(3-biphenyl-4-ylmethyl-4-hydroxy-chroman-7-yl)-cyclopentanecarboxylic acid (CP-105696), 5-[2-(2-carboxyethyl)-3-[6-(4-methoxyphenyl)-5E-hexenyl]oxyphenoxy]-valeric acid (ONO-4057) or DPC-11870,
   a 5-lipoxygenase inhibitor, such as zileuton, 6-[(3-fluoro-5-(4-methoxy-3,4,5,6-tetrahydro-2H-pyran-4-yl])phenoxymethyl-1-methyl-2-quinolone (ZD-2138), or 2,3,5-trimethyl-6-(3-pyridylmethyl)-1,4-benzoquinone (CV-6504);
   a sodium channel blocker, such as lidocaine;
   a calcium channel blocker, such as ziconotide, zonisamide, mibefradil;
   a 5-HT3 antagonist, such as ondansetron;
   a chemotherapy drug such as oxaliplatin, 5-tflorouracil, leukovorin, paclitaxel;
   a calcitonin gene related peptide (CGRP) antagonist;
   a bradykinin (BK1 and BK2) antagonist;
   a voltage gated sodium dependent channel blocker (Na_{v1.3}, Na_{v1.7}, Na_{v1.8});
   a voltage dependent calcium channel blocker (N-type, T-type);
   a P2X (ion channel type ATP receptor) antagonist;
   an acid-sensing ion channel (ASIC1a, ASIC3) antagonist;
   an angiotensin AT2 antagonist;
   a chemokine CCR2B receptor antagonist;
   a cathepsin (B, S, K) inhibitor;
   a signal receptor agonist or antagonist;
   a calcium/magnesium;
   a goshajinkigan;

and the pharmaceutically acceptable salts and solvates thereof.

Such combinations offer significant advantages, including synergistic activity, in therapy.

The composition may contain from 0.1% to 99% by weight, preferably from 10% to 60% by weight, of the active material, depending on the method of administration. The dose of the compound used in the treatment of the aforementioned disorders may vary in the usual way with the seriousness of the disorders, the weight of the sufferer, and other similar factors.

A therapeutically effective amount of a compound of formula (I) or a pharmaceutical composition thereof includes a dose range from about 0.05 mg to about 3000 mg, in particular from about 1 mg to about 1000 mg or, more particularly, from about 10 mg to about 500 mg of active ingredient in a regimen of about once a day or more than once a day, for example two, three or four times a day for an average (70 kg) human; although, it is apparent to one skilled in the art that the therapeutically effective amount for active compounds of the invention may vary as well the diseases, syndromes, conditions, and disorders being treated.

For oral administration, a pharmaceutical composition is preferably provided in the form of tablets containing about 0.01, about 10, about 50, about 100, about 150, about 200, about 250, and about 500 milligrams of the inventive compound as the active ingredient.

Advantageously, a compound of formula (I) may be administered in a single daily dose, or the total daily dosage may be administered in divided doses of two, three and four times daily.

Optimal dosages of a compound of formula (I) to be administered may be readily determined and may vary with the particular compound used, the mode of administration, the strength of the preparation, and the advancement of the disease, syndrome, condition, or disorder. In addition, factors associated with the particular subject being treated, including subject age, weight, diet and time of administration, will result in the need to adjust the dose to achieve an appropriate therapeutic level.

The above dosages are thus exemplary of the average case. There can be individual instances wherein higher or lower dosage ranges are merited of course, and such are within the scope of this invention.

Compounds of formula (I) may be administered in any of the foregoing compositions and dosage regimens or by means of those compositions and dosage regimens established in the art whenever use of a compound of formula (I) is required for a subject in need thereof.

As antagonists of the TRPM8 ion channel, the compounds of formula (I) are useful in methods for treating and preventing a disease, a syndrome, a condition, or a disorder in a subject, including an animal, a mammal and a human in which the disease, the syndrome, the condition, or the disorder is affected by the modulation of TRPM8 receptors. Such methods comprise, consist of, and consist essentially of administering to a subject, including an animal, a mammal, and a human in need of such treatment or prevention a therapeutically effective amount of a compound, salt, or solvate of formula (I). In particular, the compounds of formula (I) are useful for preventing or treating pain; diseases, syndromes, conditions, or disorders causing such pain; or pulmonary or vascular dysfunction. More particularly, the compounds of formula (I) are useful for preventing or treating inflammatory pain, inflammatory hypersensitivity conditions, neuropathic pain, anxiety, depression, and cardiovascular disease aggravated by cold, including peripheral vascular disease, vascular hypertension, pulmonary hypertension, Raynaud's disease, and coronary artery disease, by administering to a subject in need thereof a therapeutically effective amount of a compound of formula (I).

Examples of inflammatory pain include pain due to a disease, condition, syndrome, disorder, or a pain state including inflammatory bowel disease, visceral pain, migraine, post operative pain, osteoarthritis, rheumatoid arthritis, back pain, lower back pain, joint pain, abdominal pain, chest pain, labor, musculoskeletal diseases, skin diseases, toothache, pyrosis, burn, sunburn, snake bite, venomous snake bite, spider bite, insect sting, neurogenic bladder, interstitial cystitis, urinary tract infection, rhinitis, contact dermatitis/hypersensitivity, itch, eczema, pharyngitis, mucositis, enteritis, irritable bowel syndrome, Raynaud's syndrome, cholecystitis, pancreatitis, postmastectomy pain syndrome, menstrual pain, endometriosis, sinus headache, tension headache, or arachnoiditis.

One type of inflammatory pain is inflammatory hyperalgesia, which can be further distinguished as inflammatory somatic hyperalgesia or inflammatory visceral hyperalgesia. Inflammatory somatic hyperalgesia can be characterized by the presence of an inflammatory hyperalgesic state in which a hypersensitivity to thermal, mechanical and/or chemical stimuli exists. Inflammatory visceral hyperalgesia can also be characterized by the presence of an inflammatory hyperalgesic state, in which an enhanced visceral irritability exists.

Examples of inflammatory hyperalgesia include a disease, syndrome, condition, disorder, or pain state including inflammation, osteoarthritis, rheumatoid arthritis, back pain, joint pain, abdominal pain, musculoskeletal diseases, skin diseases, post operative pain, headache, toothache, burn, sunburn, insect sting, neurogenic bladder, urinary incontinence, interstitial cystitis, urinary tract infection, cough, asthma, chronic obstructive pulmonary disease, rhinitis, contact dermatitis/hypersensitivity, itch, eczema, pharyngitis, enteritis, irritable bowel syndrome, Raynaud's syndrome, inflammatory bowel diseases including Crohn's disease or ulcerative colitis.

One embodiment of the present invention is directed to a method for treating inflammatory somatic hyperalgesia in which a hypersensitivity to thermal, mechanical and/or chemical stimuli exists, comprising the step of administering to a mammal in need of such treatment a therapeutically effective amount of a compound, salt or solvate of formula (I).

A further embodiment of the present invention is directed to a method for treating inflammatory visceral hyperalgesia in which an enhanced visceral irritability exists, comprising, consisting of, and/or consisting essentially of the step of administering to a subject in need of such treatment a therapeutically effective amount of a compound, salt or solvate of formula (I).

A further embodiment of the present invention is directed to a method for treating neuropathic cold allodynia in which a hypersensitivity to a cooling stimuli exists, comprising, consisting of, and/or consisting essentially of the step of administering to a subject in need of such treatment a therapeutically effective amount of a compound, salt or solvate of formula (I).

Examples of an inflammatory hypersensitivity condition include urinary incontinence, benign prostatic hypertrophy, cough, asthma, rhinitis and nasal hypersensitivity, itch, contact dermatitis and/or dermal allergy, and chronic obstructive pulmonary disease.

Examples of a neuropathic pain include pain due to a disease, syndrome, condition, disorder, or pain state including cancer, neurological disorders, spine and peripheral nerve surgery, brain tumor, traumatic brain injury (TBI), spinal cord trauma, chronic pain syndrome, fibromyalgia, chronic fatigue syndrome, neuralgias (trigeminal neuralgia, glossopharyngeal neuralgia, postherpetic neuralgia and causalgia), lupus, sarcoidosis, peripheral neuropathy, bilateral peripheral neuropathy, diabetic neuropathy, central pain, neuropathies associated with spinal cord injury, stroke, amyotrophic lateral sclerosis (ALS), Parkinson's disease, multiple sclerosis, sciatic neuritis, mandibular joint neuralgia, peripheral neuritis, polyneuritis, stump pain, phantom limb pain, bony fractures, oral neuropathic pain, Charcot's pain, complex regional pain syndrome I and II (CRPS I/I), radiculopathy, Guillain-Barre syndrome, meralgia paraesthetica, burning-mouth syndrome, optic neuritis, postfebrile neuritis, migrating neuritis, segmental neuritis, Gombault's neuritis, neuronitis, cervicobrachial neuralgia, cranial neuralgia, geniculate neuralgia, glossopharyngeal neuralgia, migrainous neuralgia, idiopathic neuralgia, intecostal neuralgia, mammary neuralgia, Morton's neuralgia, nasociliary neuralgia, occipital neuralgia, red neuralgia, Sluder's neuralgia, sphenopalatine neuralgia, supraorbital neuralgia, vulvodynia, or vidian neuralgia.

One type of neuropathic pain is neuropathic cold allodynia, which can be characterized by the presence of a neuropathy-associated allodynic state in which a hypersensitivity to cooling stimuli exists. Examples of neuropathic cold allodynia include allodynia due to a disease, condition, syndrome, disorder or pain state including neuropathic pain or neuralgia, pain arising from spine and peripheral nerve surgery or trauma, traumatic brain injury (TBI), trigeminal neuralgia, postherpetic neuralgia, causalgia, peripheral neuropathy, diabetic neuropathy, central pain, stroke, peripheral neuritis, polyneuritis, complex regional pain syndrome I and II (CRPS I/II) and radiculopathy.

Examples of anxiety include social anxiety, post traumatic stress disorder, phobias, social phobia, special phobias, panic disorder, obsessive compulsive disorder, acute stress disorder, separation anxiety disorder, and generalized anxiety disorder.

Examples of depression include major depression, bipolar disorder, seasonal affective disorder, post natal depression, manic depression, and bipolar depression.

### General Synthesis

Throughout the instant application, the following abbreviations are used with the following meanings:
AcOH: Acetic acid
aq.: aqueous
BINAP: 2,2′-Bis(diphenylphosphino)-1,1′-binaphthyl
tBuXPhos: 2-Di-tert-butylphosphino-2′,4′,6′-triisopropylbiphenyl
CDI: Carbonyldiimidazole
Cs₂CO₃: Cesium carbonate
DABCO: 1,4-diazabicyclo[2.2.2]octane
DavePhos: 2-Dicyclohexylphosphino-2′-(N,N-dimethylamino)biphenyl
DBN: 1,5-diazabicyclo[4.3.0]non-5-ene
DBU: 1,8-Diazabicyclo[5.4.0]undec-7-ene
DCM: Dichloromethane
DEAD: Diethyl azodicarboxylate
DIPEA: Diisopropylethylamine
DMF: N,N-Dimethylformamide
DMA: N,N-Dimethylacetamide
DME: 1,2-Dimethoxyethane
DMSO: Dimethyl sulfoxide
Dess-Martin Periodinane:
1,1,1-Tris(acetyloxy)-1,1-dihydro-1,2-benziodoxol-3-(H)-one
ESI: Electrospray Ionization
Et: Ethyl
EtOAc: Ethyl acetate
EtOH: Ethanol
eq.: equivalent
FIA: Flow Injection Analysis
HPLC: High-Performance Liquid Chromatography
INT: Intermediate
IPE: Isopropyl ether
K₂CO₃: Potassium carbonate
K₃PO₄: Potassium phosphate
KO t-Bu: Potassium tert-butoxide
LC: Liquid Chromatography
LDA: Lithium diisopropylamide
LG: Leaving Group
tR: Retention Time
Me: Methyl
MeCN: Acetonitrile
MeOH: Methanol
min: minute
NaHCO₃: Sodium bicarbonate
Na₃SO₄: Sodium sulfate
Na₂S₂O₃: Sodium thiosulfate
NaO t-Bu: Sodium tert-butoxide
MHz: Megahertz
mp: melting point
MS: Mass Spectrometry
NMP: N-methyl-2-pyrrolidone
NMR: Nuclear Magnetic Resonance
Oxone (Registered Trademark): Potassium peroxymonosulfate
PG: Protecting Group
Pd₂(dba)₃: Tris(dibenzylideneacetone)dipalladium(0)
Pd(OAc)₂: Palladium (II) acetate
PdCl₂(dppf).CH₂Cl₂: [1,1′-Bis(diphenylphosphino)ferrocene]dichloropalladium (II), dichloromethane Adduct
PdCl₂(Amphos)₂: Bis(di-tert-butyl(4-dimethylaminophenyl)phosphine)dichloropalladium(II)
PEPPSI(Trademark)-IPr: [1,3-Bis(2,6-diisopropylphenyl)imidazol-2-ylidene](3-chloropyridyl)palladium(II) dichloride
Pd(PPh₃)₄: Tetrakis(triphenylphosphine)palladium (0)
POCl₃: Phosphorus(V) oxychloride
quant.: quantitative
rt: room temperature
sat.: saturated
TEA: Triethylamine
TFA: Trifluoroacetic Acid
THF: Tetrahydrofuran
THP: 2-Tetrahydropyranyl
p-TsOH: p-Toluenesulfonic acid
XPhos: 2-Dicyclohexylphosphino-2′,4′,6′-triisopropylbiphenyl
Xantphos: 4,5-Bis(diphenylphosphino)-9,9-dimethylxanthene

The term of “base” is likewise no particular restriction on the nature of the bases used, and any base commonly used in reactions of this type may equally be used here. Examples of such bases include: alkali metal hydroxides, such as lithium hydroxide, sodium hydroxide, potassium hydroxide, and barium hydroxide; alkali metal hydrides, such as lithium hydride, sodium hydride, and potassium hydride; alkali metal alkoxides, such as sodium methoxide, sodium ethoxide, and potassium t-butoxide; alkali metal carbonates, such as lithium carbonate, sodium carbonate, potassium carbonate, and cesium carbonate; alkali metal hydrogencarbonates, such as lithium hydrogencarbonate, sodium hydrogencarbonate, and potassium hydrogencarbonate; amines, such as N-methylmorpholine, triethylamine, tripropylamine, tributylamine, diisopropylethylamine, N-methylpiperidine, pyridine, 4-pyrrolidinopyridine, picoline, 2,6-di(t-butyl)-4-methylpyridine, quinoline. N,N-dimethylaniline, N,N-diethylaniline, 1,5-diazabicyclo[4.3.0]non-5-ene (DBN), 1,4-diazabicyclo[2.2.2]octane (DABCO), 1,8-diazabicyclo[5.4.0]undec-7-ene (DBU), lutidine, and colidine; alkali metal amides, such as lithium amide, sodium amide, potassium amide, lithium diisopropyl amide, potassium diisopropyl amide, sodium diisopropyl amide, lithium bis(trimethylsilyl)amide and potassium bis(trimethylsilyl)amide. Of these, triethylamine, diisopropylethylamine, DBU, DBN, DABCO, pyridine, lutidine, colidine, sodium carbonate, sodium hydrogencarbonate, sodium hydroxide, potassium carbonate, potassium hydrogencarbonate, potassium hydroxide, potassium phosphate, barium hydroxide, and cesium carbonate are preferred.

The reactions are normally and preferably effected in the presence of inert solvent. There is no particular restriction on the nature of the solvent to be employed, provided that it has no adverse effect on the reaction or the reagents involved and that it can dissolve reagents, at least to some extent. Examples of suitable solvents include, but not limited to: halogenated hydrocarbons, such as DCM, chloroform, carbon tetrachloride, and dichloroethane; ethers, such as diethyl ether, diisopropyl ether, THF, and dioxane; aromatic hydrocarbons, such as benzene, toluene and nitrobenzene; amides, such as, DMF, DMA, and hexamethylphosphoric triamide; amines, such as N-methylmorpholine, triethylamine, tripropylamine, tributylamine, diisopropylethylamine, N-methylpiperidine, pyridine, 4-pyrrolidinopyridine, N,N-dimethylaniline, and N,N-diethylaniline; alcohols, such as methanol, ethanol, propanol, isopropanol, and butanol; nitriles, such as acetonitrile and benzonitrile; sulfoxides, such as dimethyl sulfoxide (DMSO) and sulfolane; ketones, such as acetone and diethylketone. Of these solvents, including but not limited to DMF, DMA, DMSO, THF, diethylether, diisopropylether, dimethoxyethane, acetonitrile, DCM, dichloroethane and chloroform are preferred.

### EXAMPLES

The invention is illustrated in the following non-limiting examples in which, unless stated otherwise: all reagents are commercially available, all operations are carried out at room or ambient temperature, that is, in the range of about 18 to 25° C.; evaporation of solvent is carried out using a rotary evaporator under reduced pressure with a bath temperature of up to about 60° C.; reactions are monitored by thin layer chromatography (TLC) and reaction times are given for illustration only; the structure and purity of all isolated compounds are assured by at least one of the following techniques: TLC (Merck silica gel 60 F₂₅₄ precoated TLC plates or Merck NH₂ F₂₅₄ precoated HPTLC plates), mass spectrometry or nuclear magnetic resonance (NMR). Microwave reaction is conducted by Initiator+Sixty Biotage (registered trademark). Yields are given for illustrative purposes only. The column chromatography systems are conducted by Yamazen flash chromatography and Biotage (SPI, Isolera one). Flash column chromatography is carried out using Merck silica gel 60 (230-400 mesh ASTM), Fuji Silysia Chromatorex (registered trademark) DM2035 (Amino Type, 30-50 micrometer), Biotage silica (32-63 mm, KP-Sil), Biotage amino bounded silica (45-75 mm, KP-NH), Wakogel (registered trademark) C-300HGT, Hi-Flash (registered trademark) column (YAMAZEN, silica gel, 40 micrometers, 60 angstrom), Hi-Flash (registered trademark) column (YAMAZEN, amino, 40 micrometers, 60 angstrom). LC-MS analysis for intermediates and Examples are carried out by Waters 2695 Alliance HPLC with ZQ 2000 mass spectrometer and 2996 PDA detector. Analytical conditions (method-A, method-B, method-C, method-D, method-E and method-F) are as follows.

TABLE 1
Conditions for method-A, method-B, and method-C:

| Column | Waters XTerra C18, 2.1 × 30 mm, 3.5 micrometer | |
|---|---|---|
| Column temperature | 45° C. | |
| Flow rate | 0.5 mL/min | |
| PDA detection | 210-400 nm scan (Extracted wave length: 254 nm) | |
| MS detection | ESI positive & negative mods | |
| Mobile phases | A: | MeCN (HPLC grade) |
| | B: | 0.5% aqueous HCO₂H |
| | C: | 0.2% aqueous NH₃ |
| | D: | H₂O (Milli-Q water) |

TABLE 2
Method-A

| Time (min) | A(%) | B(%) | C(%) | D(%) |
|---|---|---|---|---|
| 0 | 4 | 4.8 | 4.8 | 86.4 |
| 2 | 96 | 0.2 | 0.2 | 3.6 |

run time: 4 min

TABLE 3
Method-B

| Time (min) | A(%) | B(%) | C(%) | D(%) |
|---|---|---|---|---|
| 0 | 4 | 0 | 4.8 | 91.2 |
| 2 | 96 | 0 | 0.2 | 3.8 |

run time: 4 min

TABLE 4
Method-C

| Time (min) | A(%) | B(%) | C(%) | D(%) |
|---|---|---|---|---|
| 0 | 32 | 3.4 | 3.4 | 61.2 |
| 2 | 96 | 0.2 | 0.2 | 3.6 |

run time: 4 min

TABLE 5
Conditions for method-D and method-E:

| Column | Waters SunFire C18, 4.6 × 50 mm, 5 micrometer | |
|---|---|---|
| Column temperature | 45° C. | |
| Flow rate | 0.8 mL/min | |
| PDA detection | 210-400 nm scan (Extracted wave length: 215 nm) | |
| MS detection | ESI positive & negative mode | |
| Mobile phases | A: | MeCN (HPLC grade) |
| | B: | 0.5% aqueous HCO₂H |
| | C: | 0.2% aqueous NH₃ |
| | D: | H₂O (Milli-Q water) |

TABLE 6
Method-D

| Time (min) | A(%) | B(%) | C(%) | D(%) |
|---|---|---|---|---|
| 0 | 5 | 2.5 | 2.5 | 90 |
| 0.5 | 5 | 2.5 | 2.5 | 90 |
| 3.5 | 95 | 2.5 | 2.5 | 0 |
| 4 | 95 | 2.5 | 2.5 | 0 |

run time: 4.5 min

TABLE 7
Method-E

| Time (min) | A(%) | B(%) | C(%) | D(%) |
|---|---|---|---|---|
| 0 | 5 | 0 | 5 | 90 |
| 0.5 | 5 | 0 | 5 | 90 |
| 3.5 | 95 | 0 | 5 | 0 |
| 4 | 95 | 0 | 5 | 0 |

run time: 4.5 min

The purification of compounds using HPLC (preparative LC-MS) is performed by the following apparatus and conditions.

Apparatus; Waters MS-trigger AutoPurification (trademark) system

Column; Waters XTerra C18, 19×50 mm, 5 micrometer particle

Condition A: Methanol or acetonitrile/0.01% (v/v) ammonia aqueous solution Condition B: Methanol or acetonitrile/0.05% (v/v) formic acid aqueous solution Low-resolution mass spectral data (ESI) are obtained by the following apparatus and conditions: Apparatus; Waters Alliance HPLC system on ZQ or ZMD mass spectrometer and UV detector. LC/MS/MS data are determined at the triple quadrupole mass spectrometry (AB SCIEX API4000) with HPLC (Agilent 1100 series) and autosampler (AMR CTC-PAL). NMR data are determined at 270 MHz (JEOL JNM-LA 270 spectrometer). 300 MHz (JEOL JNM-LA300) or 600 MHz (Bruker Avance 600) using deuterated chloroform (99.8% D) or dimethylsulfoxide (99.9% D) as solvent unless indicated otherwise, relative to tetramethylsilane (TMS) as internal standard in parts per million (ppm); conventional abbreviations used are: s=singlet, d=doublet, t=triplet, q=quartet, m=multiplet, br=broad, etc. Chemical symbols have their usual meanings; M (mole(s) per liter), L (liter(s)), mL (milliliter(s)), g (gram(s)), mg (milligram(s)), mol (moles), mmol (millimoles).

Each prepared compound is generally named by ChemBioDraw (Ultra, version 12.0, CambridgeSoft).

Conditions for determining HPLC retention time:
Method: QC1
Apparatus: Waters ACQUITY Ultra Performance LC with TUV Detector and ZQ mass spectrometer

Column: Waters ACQUITY C18, 2.1×100 mm, 1.7 micrometer particle size
Column Temperature: 60° C.
Flow rate: 0.7 mL/min
Run time: 3 min
UV detection: 210 nm
MS detection: ESI positive/negative mode
Mobile phases:
   A1: 10 mM Ammonium acetate
   B1: acetonitrile
Gradient program:

TABLE 8

| Time (min) | A1(%) | B1(%) |
|---|---|---|
| 0 | 95 | 5 |
| 0.1 | 95 | 5 |
| 1.8 | 5 | 95 |
| 2.3 | 95 | 5 |

Method: QC2
Apparatus: Waters 2795 Alliance HPLC with ZQ2000 mass spectrometer and 2996 PDA Detector
Column: XBridge C18, 2.1×50 mm, 3.5 micrometer particle size
Column Temperature: 45° C.
Flow rate: 1.2 mL/min
Run time: 4.5 min
UV detection: 210-400 nm scan
MS detection: ESI positive/negative mode
Mobile phases:
   A: Water
   B: MeCN
   C: 1% aqueous HCO₂H solution
   D: 1% aqueous NH₃ solution
Gradient program:

TABLE 9

| Time (min) | A (%) | B (%) | C (%) | D (%) |
|---|---|---|---|---|
| 0 | 85 | 10 | 2.5 | 2.5 |
| 0.2 | 85 | 10 | 2.5 | 2.5 |
| 3.2 | 0 | 95 | 2.5 | 2.5 |
| 3.7 | 0 | 95 | 2.5 | 2.5 |
| 3.71 | 85 | 10 | 2.5 | 2.5 |
| 4.5 | 85 | 10 | 2.5 | 2.5 |

All of the azaspiro derivatives of the formula (I) can be prepared by the procedures described in the general methods presented below or by the specific methods described in the Example synthesis part and Intermediate synthesis part, or by routine modifications thereof. The present invention also encompasses any one or more of these processes for preparing the azaspiro derivatives of formula (I), in addition to any novel intermediates used therein.

In the following general methods, descriptors are as previously defined for the azaspiro derivatives of the formula (I) unless otherwise stated.

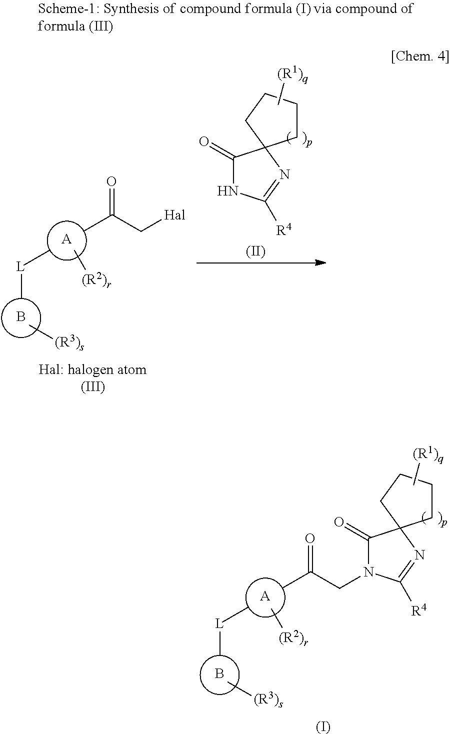

In this scheme-1, an imidazolinone compound of the general formula (I) can be prepared by the N-alkylation reaction of an imidazolinone compound of formula (II) with the alpha-haloketone compound of formula (III) in the presence of a base in an inert solvent. A preferred base is selected from, for example, but not limited to: an alkali or alkaline earth metal hydroxide, alkoxide, carbonate, halide or hydride, such as sodium hydroxide, potassium hydroxide, sodium methoxide, sodium ethoxide, potassium tert-butoxide, cesium carbonate, sodium carbonate, potassium carbonate, potassium phosphate, potassium fluoride, sodium hydride or potassium hydride; or an amine such as TEA, tributylamine, diisopropylethylamine, 2,6-lutidine, pyridine or 4-dimethylaminopyridine. Examples of suitable inert aqueous or non-aqueous organic solvents include: ethers, such as THF or 1,4-dioxane; acetone N,N-Dimethylfomamide; DMSO; halogenated hydrocarbons, such as DCM, 1,2-dichloroethane or chloroform; and pyridine; or mixtures thereof. The reaction can be carried out at a temperature in the range of from −80° C. to 200° C., preferably in the range of from −10° C. to 150° C. Reaction times are, in general, from 10 minutes to 4 days, preferably from 10 minutes to 24 h. A microwave oven may optionally be used to increase reaction rates.

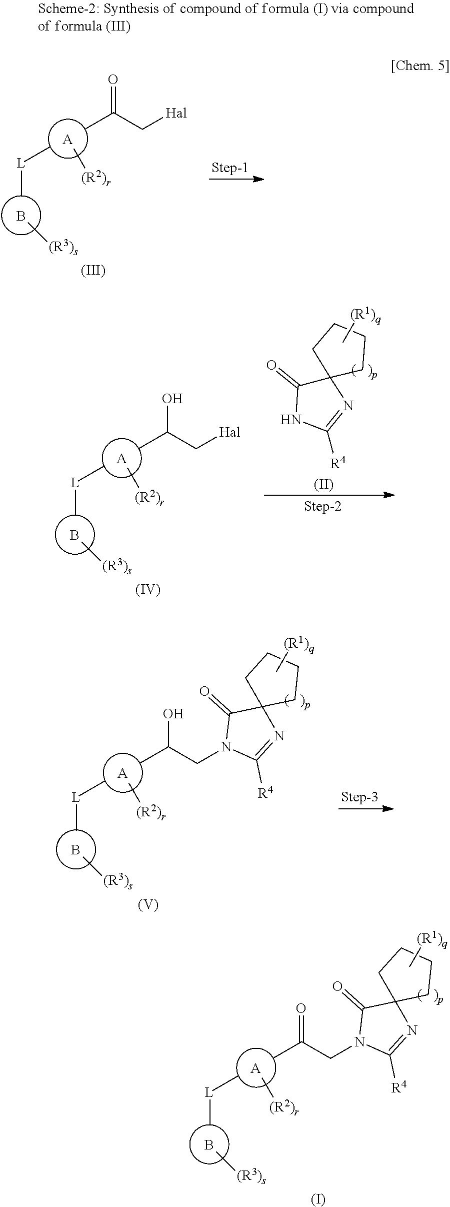

In scheme-2, a compound of the general formula (IV) can be prepared from a compound (i) using a suitable reduction reagent (for example, sodium borohydride) in an inert solvent (for example, methanol). Then, a compound of the general formula (V) can be prepared from a compound (IV) according to the N-alkylation described in the generally synthetic method in scheme-1. Finally, a compound of the general formula (I) can be prepared from a compound (V) using a suitable oxidation reagent (for example, Dess-Martin reagent) in an inert solvent (for example, dichloromethane).

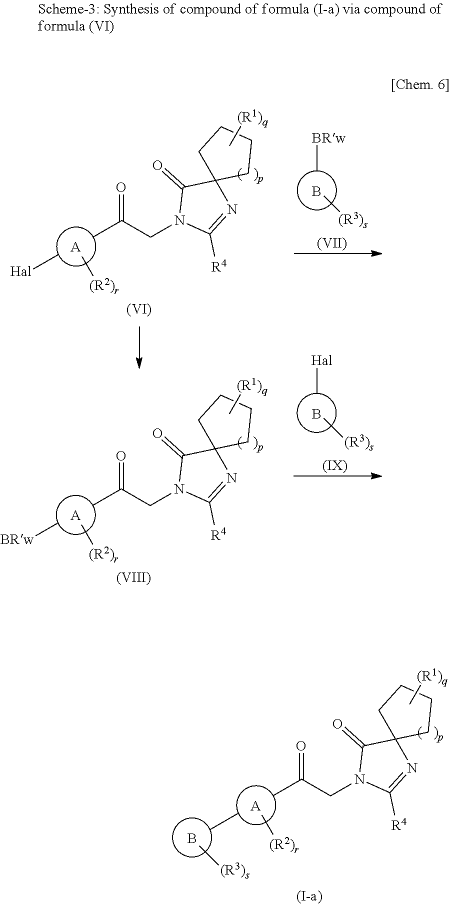

In scheme-3, a compound of the general formula (I-a) can be prepared by the cross coupling reaction of a halide compound of formula (VI) with a boronic (or boronic ester) compound of formula (VII) in organic solvent or water-organic co-solvent mixture under coupling conditions in the presence of a suitable transition metal catalyst and in the presence or absence of a base. In a presentation of BR′_{w}, R′ means OH, O-lower alkyl or fluorine, and w is 2 or 3, B is boron atom. As the concrete representation of substituent, B(OH)₂, B(O-lower alkyl)₂, B(lower alkyl)₂, potassium trifluoroborate (BF_{3′})(BF₃K) are described, but when B(O-lower alkyl)₂ may form the cyclic ring between the lower alkyl groups. Furthermore, a compound of the general formula (I-a) can also be prepared by the same cross coupling reaction from a halide compound of formula (IX) with a boronic (or boronic ester) compound of formula (VIII) converted from the halide compound of formula (VI). The boronic (or boronic ester) compounds of formula (VII) and (VI) are utilized as the isolated reagents or the reagents generated in in situ for the cross coupling reaction.

Examples of suitable transition metal catalysts include: tetrakis(triphenylphosphine)palladium(0), bis(triphenylphosphine)palladium(II) chloride, copper(0), copper(I) acetate, copper(l) bromide, copper(I) chloride, copper(I) iodide, copper(I) oxide, copper(I) trifluoromethanesulfonate, copper (II) acetate, copper(l) bromide, copper(U) chloride, copper(II) iodide, copper(H) oxide, copper(II) trifluoromethanesulfonate, palladium(I) acetate, palladium(II) chloride, bis(acetonitrile)dichloropalladium(II), bis(dibenzylideneacetone)palladium(0), tris(dibenzylideneacetone)dipalladium(0) and ([1,1′-bis(diphenylphosphino)ferrocene]palladium(II) dichloride. Preferred catalysts are tetrakis(triphenylphosphine)palladium(0), bis(triphenylphosphine)palladium(II) chloride, palladium(II) acetate, palladium(I) chloride, bis(acetonitrile)dichloropalladium(0), bis(dibenzylideneacetone)palladium(0), tris(dibenzylideetoneaetone)dipalladium(0) and [1,1-bis(diphenylphosphino)ferrocene]palladium(II) dichloride.

Examples of suitable organic solvent for the anhydrous solvent and the water-organic co-solvent mixture include: THF; 1,4-dioxane; DME; DMF; acetonitrile; alcohols, such as methanol or ethanol; halogenated hydrocarbons, such as DCM, 1,2-dichloroethane, chloroform or carbon tetrachloride; and diethylether. This reaction can be carried out in the presence or absence of a base such as potassium hydroxide, sodium hydroxide, lithium hydroxide, sodium bicarbonate, sodium carbonate, potassium carbonate and potassium phosphate. This reaction can be carried out in the presence of a suitable additive agent. Examples of such additive agents include: triphenylphosphine, tri-tert-butylphosphine, 1,1′-bis(diphenylphosphino)ferrocene, tri-2-furylphosphine, tri-o-tolylphosphine, 2-(dichlorohexylphosphino)biphenyl, triphenylarsine, tetrabutylammonium chloride, tetrabutylammonium fluoride, lithium acetate, lithium chloride, triethylamine, potassium or sodium methoxide, sodium hydroxide, cesium carbonate, tripotassium phosphate, sodium carbonate, sodium bicarbonate, and/or sodium iodide. The reaction can be carried out at a temperature of from 0° C. to 200° C., more preferably from 20° C. to 150° C. Reaction times are, in general, from 5 minutes to 96 h, more preferably from 30 minutes to 24 h. In an alternative case, the reaction can be carried out in a microwave system in the presence of a base in an inert solvent. The reaction can be carried out at a temperature in the range of from 100° C. to 200° C., preferably in the range of from 120° C. to 150° C. Reaction times are, in general, from 10 minutes to 3 h, preferably from 15 minutes to 1 h. Other than a Suzuki-Miyaura cross coupling shown above, Stifle cross coupling reaction using trialkyltin instead of BR′_{w} substituent, and Negishi coupling reaction using zinc-halogen, wherein as a halogen, chlorine, bromine, iodide are cited, instead of BR′_{w} substituent can be used.

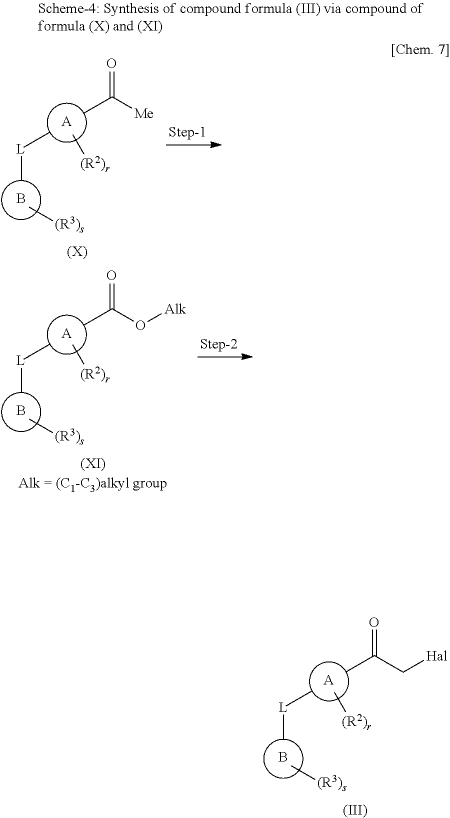

In the step-1 of scheme-4, an alpha-haloketone compound of the general formula (III) can be prepared by the alpha-halogenation reaction (Hal=Cl, Br, I) of compound (X) using an appropriate halogenation reagent. As an appropriate halogenation reagent, for example, bromine, chlorine, iodide, sulfuryl chloride, hydrogen bromine, N-bromosuccinimide (NBS), copper (II) bromide, 5,5-dibromo-2,2-dimethyl-4,6-dioxo-1,3-dioxane, trimethylphenylammonium tribromide, benzyltrimethylammonium tribromide, and benzyltrimethylammonium dichloroiodate are cited. As an appropriate organic solvent, for example, acetic acid, 25% hydrogen bromide-acetic acid solution, 48% hydrogen bromide solution, carbon disulfide, diethyl ether, tetrahydrofuran, N,N-dimethylformamide (DMF), halogenated hydrocarbon such as dichloromethane, 1,2-dichloroethane, chloroform, carbon tetrachloride can be used. The reaction period is about 5 minutes to 96 h, and is generally about 30 minutes to 24 h. The reaction temperature is about 0° C. to 250° C., and is generally about 30° C. to 150° C. Further, in the step-2 of scheme-4, alpha-haloketone compound of the general formula (UI) can also be prepared from an ester compound (XI) according to the procedure described in Tetrahedron Letters, 38, 3175, 1997. Typically, compound of formula (III) is prepared by the reaction with an ester compound (XI) under the condition of iodochloromethane and lithium diisopropylamide (LDA) in tetrahydrofuran (THF) at −78° C.

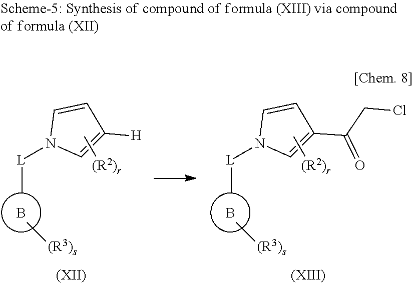

In scheme-5, an alpha-haloketone compound of the general formula (XIII) can be prepared by the Friedel-Crafts reaction of a pyrrole compound (XII) using chloroacetyl chloride and appropriate Lewis acid (for example, aluminum chloride) in an inert solvent (for example, dichloromethane).

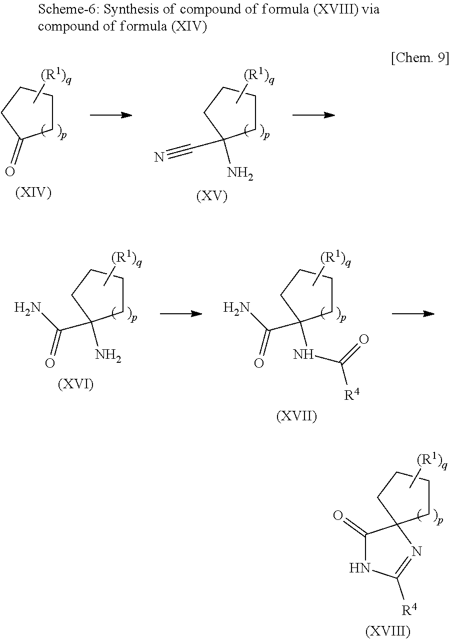

In Scheme-6, an aminocarboxamide compound of the general formula (XVI) can be prepared from Strecker reaction of a ketone compound of the general formula (XIV) by using potassium cyanide and ammonium chloride in methanol and 28% ammonia aqueous solution, followed by the acidic hydrolysis of an aminonitrile compound of the general formula (XV) by using sulfuric acid according to Synthetic Communications 35 (15), 2677-2684 (2003). Further, an imidazolinone compound of formula (XVIII) can be prepared from the acylation of an aminocarboxamide compound of the general formula (XVI) by the general condition, followed by the cyclization of the compound of the general formula (XVII) of under the acidic condition.

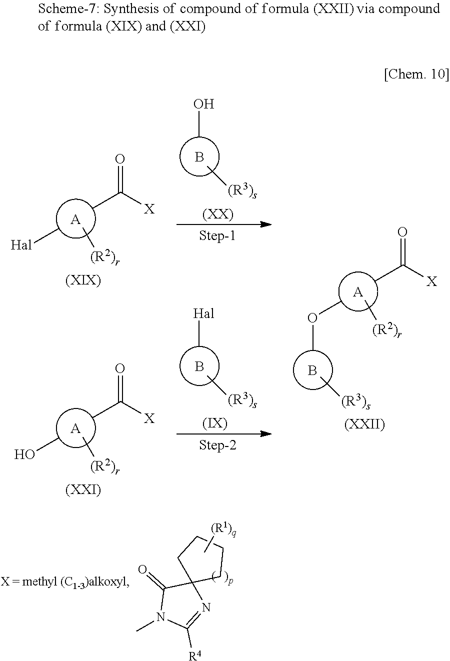

In scheme-7, a compound of the general formula (XXII) can be prepared by the reaction of a halide compound of formula (XIX) with compound of formula (XX) (step-1). Alternatively the compound of the general formula (XXII) can be also prepared by the reaction of a phenol compound of formula (XXI) with compound of formula (IX) (step-2) by using the selected procedure from palladium coupling reaction, nucleophilic substitution reaction and Ullmann reaction. The coupling reaction can be carried out by the combination of a suitable palladium catalyst, ligand and base in organic solvent or water-organic co-solvent mixture. Examples of suitable transition metal catalysts include: palladium(II) acetate, tris(dibenzylideneacetone)dipalladium(0) and [1,3-bis(2,6-diisopropylphenyl)imidazol-2-ylidene](3-chloropyridyl)palladium(II) dichloride. Examples of suitable organic solvent for the anhydrous solvent and the water-organic co-solvent mixture include: THF; DME; 1,4-dioxane; DMF; acetonitrile and alcohols, such as methanol, ethanol and tert-butyl alcohol. Examples of suitable base include: sodium bicarbonate, sodium carbonate, potassium carbonate, cesium carbonate, potassium phosphate, sodium tert-butoxide and potassium tert-butoxide. This reaction can be carried out in the presence of a suitable ligand agent. Examples of such ligand agents include: 2,2′-bis(diphenylphosphino)-1,1′-binaphthyl(BINAP), 2-dicyclohexylphosphino-2′-(N,N-dimethylamino)biphenyl(DavePhos), 4,5-bis(diphenylphosphino)-9,9-dimethylxanthene(Xantphos) and 2-dicyclohexylphosphino-2′,4′,6′-triisopropylbiphenyl(XPhos). The nucleophilic substitution reaction can be carried out in organic solvent or water-organic co-solvent mixture under coupling conditions in the presence of a base. Examples of suitable organic solvent include N,N-dimethylformamide, dimethylsulfoxide and N-methyl-2-pyrrolidinone. Examples of suitable base include sodium carbonate, potassium carbonate, cesium carbonate, potassium phosphate, sodium hydride, sodium tert-butoxide and potassium tert-butoxide. Furthermore, the Ullmann reaction can be carried out under the coupling conditions by using a suitable copper reagent, ligand and base in organic solvent. As an appropriate copper reagent, for example, copper (I) iodide, copper (I) bromide, and copper (I) chloride can be used. As an appropriate ligand and base, for example, ligand such as N,N-dimethylglycine, L-proline, N,N′-dimethylethylenediamine and trans-N,N′-dimethylcyclohexane-1,2-diamine and base such as sodium carbonate, potassium carbonate and cesium carbonate can be used. Examples of suitable organic solvent include THF, 1,4-dioxane, N,N-dimethylformamide, dimethylsulfoxide and N-methyl-2-pyrrolidinone. These reaction can be carried out at a temperature of from 20° C. to 200° C., more preferably from 100° C. to 160° C. Reaction times are, in general, from 5 minutes to 96 h, more preferably from 30 minutes to 24 h. In an alternative case, the reaction can be carried out in a microwave system in the presence of a base in an inert solvent. The reaction can be carried out at a temperature in the range of from 100° C. to 200° C., preferably in the range of from 120° C. to 150° C. Reaction times are, in general, from 10 minutes to 3 h, preferably from 15 minutes to 1 h.

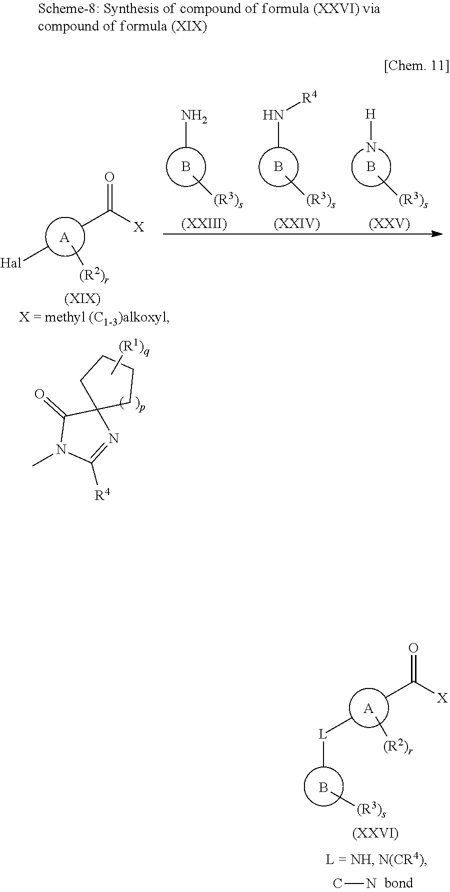

In scheme-8, a compound of the general formula (XXVI) can be prepared by the reaction of a halide compound of formula (XIX) with compound of formula (XXIII), (XXIV) or (XXV) by using the selected procedure from palladium coupling reaction, nucleophilic substitution reaction or the Ullmann reaction according to the general synthetic method in scheme-7, according to the generally synthetic method in scheme-7.

Preparation of Intermediate

### Intermediate-1-1-A (INT-1-1-A): 2-methyl-1,3-diazaspiro[4.5]dec-1-en-4-one

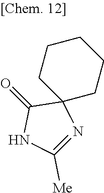 [SMILES: CC1=NC2(CCCCC2)C(=O)N1]

A mixture of 1-acetamidocyclohexanecarboxamide (130 mg, 0.706 mmol) and TPA (0.109 mL, 1.41 mmol) in toluene (10 mL) was refluxed azeotropically for 1 day. The mixture was evaporated to give the titled compound (113 mg, 96% yield) as an off-white solid.

¹H-NMR (270 MHz, DMSO-d₆): delta 2.31 (s, 3H), 1.72-1.25 (m, 10H), the signal due to NH is not observed.

MS (ESI) m/z: 167.2 (M+H)⁺.

### Intermediate-1-2-A (INT-1-2-A): 8,8-difluoro-2-methyl-1,3-diazaspiro[4.5]dec-1-en-4-one

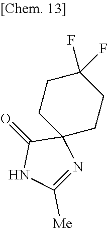 [SMILES: CC1=NC2(CCC(F)(F)CC2)C(=O)N1]

### <Step-1>: Intermediate-1-2-1 (INT-1-2-1): 1-amino-4,4-difluorocyclohexanecarboxamide

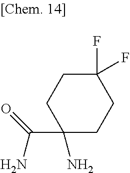 [SMILES: NC(=O)C1(N)CCC(F)(F)CC1]

To a stirred concentrated sulfuric acid (5.0 g) in an ice-cooled water bath was added dropwise a solution of 1-amino-4,4-difluorocyclohexanecarbonitrile (2.50 g, 15.61 mmol) in DCM (15 mL), maintaining the internal temperature below 15° C. After stirring at ambient temperature for 1 h, then h at 40° C., the reaction was quenched with ice-water and the mixture was basified to pH>8 with 28% ammonia aqueous solution. The mixture was extracted with EtOAc and the combined solution was washed with brine, dried over sodium sulfate, filtered and the filtrate was concentrated in vacuo to give the title compound (2265 mg, 81% yield) as a slightly yellow solid.

¹H-NMR (270 MHz, DMSO-d₆): delta 7.45 (br.s, 1H), 6.99 (br.s, 1H), 2.25-1.70 (m, 8H), 1.55-1.40 (m, 2H).

MS (FIA) m/z: 179.1 (M+H)⁺.

### <Step-2>: Intermediate-1-2-2 (INT-1-2-2): 1-acetamido-4,4-difluorocyclohexanecarboxamide

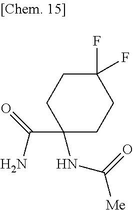 [SMILES: CC(=O)NC1(C(N)=O)CCC(F)(F)CC1]

To a stirred solution of INT-1-2-1 (2076 mg, 11.65 mmol) and triethylamine (3.25 mL, 23.30 mmol) in DCM (80 mL) was added acetyl chloride (0.911 mL, 12.82 mmol) via a syringe at rt. After stirring at rt for 15 h, the mixture was diluted with minimum DCM and the precipitated solid was collected to give the titled compound (1345 mg, 52% yield) as a white solid.

¹H-NMR (270 MHz, DMSO-d₆): delta 7.83 (br.s, 1H), 7.14 (br.s, 1H), 6.91 (br.s, 1H), 2.20-1.75 (m, 8H), 1.89 (s, 3H).

MS (FIA) m/z: 219.3 (M−H)⁻.

### <Step-3>: Intermediate-1-2-A (INT-1-2-A): 2-ethyl-8,8-difluoro-1,3-diazaspiro[4.5]dec-1-en-4-one

A mixture of INT-1-2-2 (600 mg, 2.72 mmol) in AcOH (10 mL) and TFA (1 mL) was irradiated in a microwave reactor (Biotage Initiator) at 140° C. for 90 min. After the removal of solvent, the residue was dissolved in methanol. The crude product was purified by short column chromatography on amine silica gel eluting with DCM-methanol (1:1) to give the titled compound (287 mg, 52% yield) as a white solid.

¹H-NMR (270 MHz, DMSO-d₆): delta 2.20-1.90 (m, 4H), 2.07 (s, 3H), 1.85-1.60 (m, 2H), 1.56-1.40 (m, 214).

MS (FIA) m/z: 203.1 (M+H)⁺.

### Intermediate-1-3-A (INT-1-3-A): 2-ethyl-8,8-difluoro-1,3-diazaspiro[4.5]dec-1-en-4-one

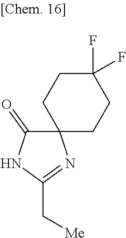 [SMILES: CCC1=NC2(CCC(F)(F)CC2)C(=O)N1]

### <Step-1>: Intermediate-1-3-1 (INT-1-3-1): 4,4-difluoro-1-propionamidocyclohexanecarboxamide

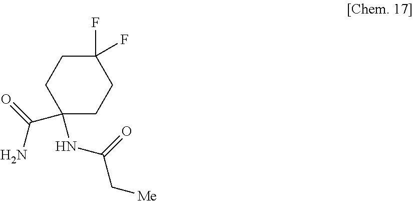 [SMILES: CCC(=O)NC1(C(N)=O)CCC(F)(F)CC1]

To a stirred solution of INT-1-2-1 (2260 mg, 12.68 mmol) and triethylamine (3.54 mL, 25.4 mmol) in DCM (80 mL) was added propionyl chloride (1.21 mL, 13.95 mmol) via a syringe at rt. After stirring at it for 15 h, the mixture was diluted with minimum DCM and the precipitated solid was filtered to give the titled compound (1609 mg, 54% yield) as a white solid.

¹H-NMR (270 MHz, DMSO-d₆): delta 7.73 (br.s, 1H), 7.10 (br.s, 1H), 6.90 (br.s, 1H), 2.19 (q, J=7.9 Hz, 2H), 2.15-2.05 (m, 2H), 2.02-1.80 (m, 6H), 0.98 (t, J=7.9 Hz, 3H).

MS (FIA) m/z: 233.2 (M−H)−.

### <Step-2>: Intermediate-1-3-A (INT-1-3-A): 8,8-difluoro-2-methyl-1,3-diazaspiro[4.5]dec-1-en-4-one

A mixture of INT-1-3-1 (800 mg, 3.42 mmol) in AcOH (12 mL) and TFA (2.4 mL) was irradiated in a microwave reactor (Biotage Initiator) at 150° C. for 60 min. After the removal of solvent, the residue was dissolved in methanol. The crude product was purified by short column chromatography on amine silica gel eluting with DCM-methanol (1:1) to give the titled compound (617 mg, 84% yield, chemical purity 70%) as a white solid.

¹H-NMR (270 MHz, DMSO-d₆): delta 2.38 (q, J=7.3 Hz, 2H), 2.30-1.60 (m, 6H), 1.57-1.42 (m, 2H), 1.13 (t, J=7.3 Hz, 3H), the signal due to NH is not observed. MS (FIA) m/z: 217.1 (M+H)⁺.

### Intermediate-2-1 (INT-2-1): 2-chloro-1-(4-(pyridazin-3-yloxy)phenyl)ethanone

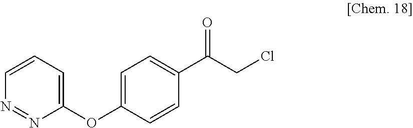 [SMILES: O=C(CCl)c1ccc(Oc2cccnn2)cc1]

### <Step-1>: Intermediate-2-1-1 (INT-2-1-1): 1-(4-(pyridazin-3-yloxy)phenyl)ethanone

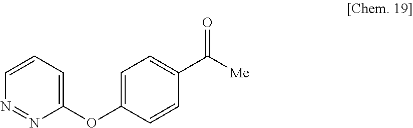 [SMILES: CC(=O)c1ccc(Oc2cccnn2)cc1]

A mixture of 1-(4-hydroxyphenyl)ethanone (283 mg, 2.08 mmol), 3-chloropyridazine (238 mg, 2.08 mmol) and potassium carbonate (574 mg, 4.16 mmol) in DMF (5 mL) was irradiated in a microwave reactor (Biotage Initiator) for 60 min. at 140° C. After cooling, the reaction mixture was filtered through Celite pad and the filtrate was washed with EtOAc. The filtrate was diluted with water and the organic layer was separated. After the extraction of the aqueous layer with EtOAc, the combined organic layers were washed with brine, dried over sodium sulfate, filtered and the filtrate was concentrated in vacuo. The residue was purified by column chromatography (Biotage) on silica gel (25 g) eluting with 10-80% EtOAc in DCM to give the titled compound (58 mg, 13% yield) as a white solid.

¹H-NMR (270 MHz, DMSO-d₆): delta 9.07 (dd, J=4.6, 1.3 Hz, 1H), 8.06 (d, J=8.5 Hz, 2H), 7.84 (dd, J=9.2, 4.6 Hz, 1H), 7.59-7.55 (m, 1H), 7.36 (d, J=8.5 Hz, 2H), 2.60 (s, 3H).

MS (ESI) m/z: 215.1 (M+H)⁺.

### Intermediate-2-1 (INT-2-1)

### 2-chloro-1-(4-(pyridazin-3-yloxy)phenyl)ethanone

A mixture of INT-2-1-1 (1.50 g, 7.00 mmol) and benzyltrimethylammonium dichloroiodate (3.66 g, 10.5 mmol) in THF (30 mL) was stirred at 75° C. for 4 h. The mixture was diluted with EtOAc and water. After the extraction twice, the combined solution was washed with 10% Na₂S₂O₃ aq. solution then brine, dried over magnesium sulfate, and filtered. The filtrate was concentrated in vacuo to give the crude product (dark brown solid). The residual solid was purified by column chromatography (Biotage) on silica gel (100 g) eluting with 20-60% EtOAc in hexane to give the titled compound (1.14 g, 66% yield) as a yellow solid.

¹H-NMR (270 MHz, DMSO-d₆): delta 9.08 (dd, J=4.6, 1.3 Hz, 1H), 8.08 (d, J=8.5 Hz, 2H), 7.84 (dd, J=8.5, 4.6 Hz, 1H), 7.61-7.57 (m, 1H), 7.39 (d, J=8.5 Hz, 2H). MS (ESI) m/z: 249.1 (M+H)⁺.

### Intermediate-2-2 (INT-2-2): 2-bromo-1-(2-methyl-1H-benzo[d]imidazol-1-yl)phenyl)ethanone⋅hydrobromide

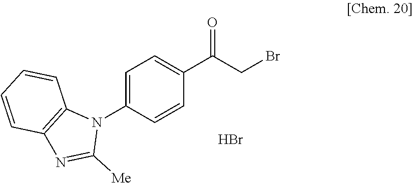 [SMILES: Cc1nc2ccccc2n1-c1ccc(C(=O)CBr)cc1 ; Br]

To a stirred solution of 1-(4-(2-methyl-1H-benzo[d]imidazol-1-yl)phenyl)ethanone (130 mg, 0.519 mmol) in 25% HBr—AcOH (3 mL) was added bromine (83 mg, 0.519 mmol). The resulting orange solution was stirred at 60° C. for 1.5 h. After cooling, the solvent was removed by nitrogen flow. The residue solid was triturated with IPE to give the titled compound (202 mg, 95% yield) as an orange solid.

¹H-NMR (270 MHz, CD₃OD): delta 8.39 (d, J=8.6 Hz, 2H), 7.86 (d, J=8.6 Hz, 2H), 7.68-7.58 (m, 4H), 4.78 (s, 2H), 2.79 (s, 3H).

MS (ESI) m/z: 331.1 (M+H)⁺.

### Intermediate-2-3 (INT-2-3): 2-bromo-1-(4-(2-methyl-3H-imidazo[4,5-b]pyridin-3-yl)phenyl)ethanone⋅hydrobromide

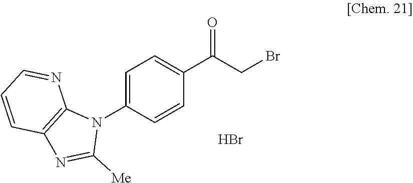 [SMILES: Cc1nc2cccnc2n1-c1ccc(C(=O)CBr)cc1 ; Br]

### <Step-1>: Intermediate-2-3-1 (INT-2-3-1): 1-(4-((2-nitrophenyl)amino)phenyl)ethanone

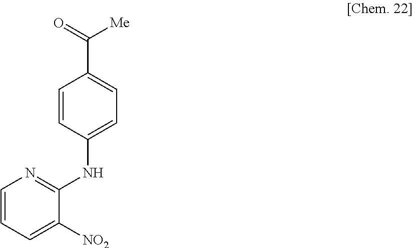 [SMILES: CC(=O)c1ccc(Nc2ncccc2[N+](=O)[O-])cc1]

A mixture of 2-chloro-3-nitropyridine (951 mg, 6.00 mmol), 1-(4-aminophenyl)ethanone (811 mg, 6.00 mmol), sodium iodide (90 mg, 0.60 mmol), racemic-BINAP (224 mg, 0.36 mmol), palladium acetate (81 mg, 0.36 mmol) and potassium carbonate (1659 mg, 12.0 mmol) in toluene (30 mL) was heated at 100° C. for 20 h. After cooling to rt, the mixture was diluted with EtOAc and water and filtered through a pad of celite. The filter cake was washed with EtOAc and the filtrate and washings were washed with brine, dried over sodium sulfate, and filtered. The filtrate was concentrated in vacuo to give the crude product, which was purified by column chromatography (Biotage) on silica gel (100 g) eluting with 3-5% ethyl acetate in DCM to give the titled compound (1273 mg, 82% yield) as a reddish yellow solid.

¹H-NMR (270 MHz, CDCl₃): delta 10.36 (br.s, 1H), 8.62-8.54 (m, 2H), 8.05-7.96 (m, 2H), 7.88-7.80 (m, 2H), 7.00-6.92 (m, 1H), 2.61 (s, 3H).

MS (ESI) m/z: 258.1 (M+H)⁺.

### <Step-2>: Intermediate-2-3-2 (INT-2-3-2): 1-(4-((2-aminophenyl)amino)phenyl)ethanone

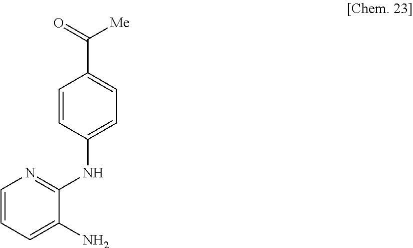 [SMILES: CC(=O)c1ccc(Nc2ncccc2N)cc1]

A mixture of INT-2-3-1 (2.6 g, 10.11 mmol), iron (3.39 g, 60.6 mmol) and solid ammonium chloride (1.62 g, 30.3 mmol) in EtOH/water (4/1 v/v)(50 mL) was heated at reflux for 2.5 h. After cooling to rt, the reaction mixture was filtered through a pad of Celite, and the filtrate was concentrated. The residue was partitioned between EtOAc and 2N NaOH aq. solution. The organic layer was washed with brine, dried over sodium sulfate, and filtered. The filtrate was concentrated in vacuo to give the titled compound (2.22 g, 97% yield) as a brown solid.

¹H-NMR (270 MHz, DMSO-ds): delta 8.28 (s, 1H), 7.85 (d, J=8.5 Hz, 2H), 7.69 (d, J=8.5 Hz, 2H), 7.57 (dd, J=4.6, 1.3 Hz, 1H), 6.98 (dd, J=7.9, 1.3 Hz, 1H), 6.75 (dd, J=7.9, 4.6 Hz, 1H), 5.20 (s, 2H), 2.46 (s, 3H).

MS (ESI) m/z: 228.1 (M+H)⁺.

### <Step-3>: Intermediate-2-3-3 (INT-2-3-3): N-(2-((4-acetylphenyl)amino)phenyl)acetamide

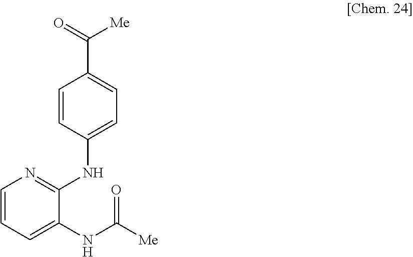 [SMILES: CC(=O)Nc1cccnc1Nc1ccc(C(C)=O)cc1]

A mixture of INT-2-3-2 (2.22 g, 9.77 mmol), acetic anhydride (1.05 g, 10.26 mmol) and triethylamine (2.97 g, 29.3 mmol) in DCM (40 mL) was stirred at it for 4 h. The mixture was concentrated in vacuo to give a titled compound, which was used for the next step without the further purification.

¹H-NMR (270 MHz, DMSO-d₆): delta 9.56 (s, 1H), 8.62 (s, 1H), 8.08 (d, J=3.3 Hz, 1H), 7.89 (d, J=9.2 Hz, 2H), 7.76-7.71 (m, 3H), 6.95 (dd, J=4.6, 7.3 Hz, 1H), 2.50 (s, 3H), 2.12 (s, 3H).

MS (ESI) m/z: 270.1 (M+H)⁺.

### <Step-4>: Intermediate-2-3-4 (INT-2-3-4): 1-(4-(2-methyl-3H-imidazo[4,5-b]pyridin-3-yl)phenyl)ethanone

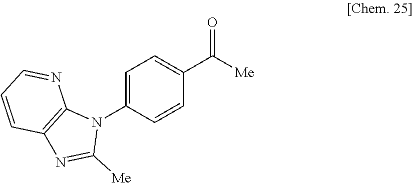 [SMILES: CC(=O)c1ccc(-n2c(C)nc3cccnc32)cc1]

A solution of INT-2-3-3 (2.63 g, 9.77 mmol) in acetic acid (40 mL) was stirred at 100° C. for 15 h. After cooling, the reaction mixture was concentrated in vacuo. The residual oil was diluted with EtOAc and basified to pH>8 with sat. NaHCO₃ aq. solution. After the extraction with EtOAc, the combined organic layers were washed with brine, dried over sodium sulfate, and filtered. The filtrate was concentrated in vacuo. The residual solid was purified by column chromatography (Biotage) on silica gel (100 g) eluting with 10-100% ethyl acetate in DCM to give the titled compound (2.32 g, 95% yield) as a light brown solid.

¹H-NMR (270 MHz, DMSO-d₆): delta 8.25 (dd, J=5.3, 1.3 Hz, 1H), 8.18 (d, J=8.5 Hz, 2H), 8.06 (dd, J=7.9, 1.3 Hz, 1H), 7.77 (d, J=8.5 Hz, 2H), 7.32 (dd, J=7.9, 5.3 Hz, 1H), 2.68 (s, 3H), 2.53 (s, 3H).

MS (ESI) m/z: 252.1 (M+H)⁺.

### <Step-5>: Intermediate-2-3 (INT-2-3): 2-bromo-1-(4-(2-methyl-3H-imidazo[4,5-b]pyridin-3-yl)phenyl)ethanone⋅hydrobromide

To a stirred solution of INT-2-3-4 (250 mg, 0.995 mmol) in 25% HBr—AcOH (5 mL) was added bromine (159 mg, 0.995 mmol). The resulting orange solution was stirred at rt for 1 h.

After the removal of solvent by nitrogen flow, the residual solid was triturated with MeOH/IPE (1/1 w/w) to give the titled compound (462 mg, 0.939 mmol) as a yellow solid.

¹H-NMR (270 MHz, DMSO-d₆): delta 8.51 (d, J=3.3 Hz, 1H), 8.37 (d, J=7.9 Hz, 1H), 8.30 (d, J=8.5 Hz, 2H), 7.89 (d, J=8.5 Hz, 2H), 7.65-7.59 (m, 1H), 5.07 (s, 2H), 2.72 (s, 3H).

MS (ESI) m/z: 331.9 (M+H)⁺.

### Intermediate-2-4 (INT-2-4): 2-bromo-1-(4-(4-methylpyridazin-3-yl)phenyl)ethanone

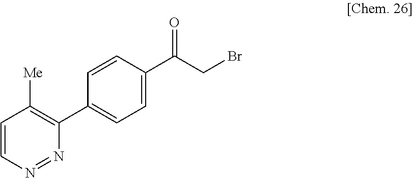 [SMILES: Cc1ccnnc1-c1ccc(C(=O)CBr)cc1]

### <Step-1>: Intermediate-2-4-1 (INT-2-4-1): 1-(4-(4-methylpyridazin-3-yl)phenyl)ethanone

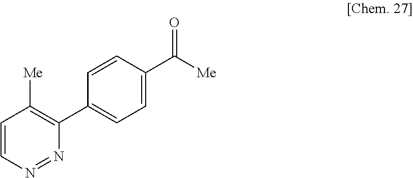 [SMILES: CC(=O)c1ccc(-c2nnccc2C)cc1]

A mixture of 3-chloro-4-methylpyridazine (1200 mg, 9.33 mmol), (4-acetylphenyl)boronic acid (1530 mg, 9.33 mmol), PdCl₂(dppf)-CH₂Cl₂ adduct (381 mg, 0.467 mol) in 1,4-dioxane (15 mL) and sat. NaHCO₃ aqueous solution (15 mL) was stirred at 80° C. for 3 h. After cooling to rt, the mixture was diluted with EtOAc (200 mL)-water (20 mL) and filtered through a pad of Celite. After washing of filter cake with EtOAc, the combined solution was washed with water, brine, dried over sodium sulfate and filtered. The filtrate was concentrated in vacuo to give the crude product (dark yellow oil), which was purified by column chromatography on silica gel (100 g) eluting with 50-100% ethyl acetate in hexane to give the titled compound (1.24 g, 62% yield) as a slightly yellow solid.

¹H-NMR (270 MHz, CDCl₃): delta 9.08 (d, J=5.3 Hz, 1H), 8.11 (d, J=8.6 Hz, 2H), 7.71 (d, J=8.6 Hz, 2H), 7.41 (d, J=5.3 Hz, 1H), 2.68 (s, 3H), 2.39 (s, 3H). MS (ESI) m/z: 213.1 (M+H)⁺

### <Step-2>: Intermediate-2-4 (INT-2-4): 2-bromo-1-(4-(4-methylpyridazin-3-yl)phenyl)ethanone

To a stirred mixture of INT-2-4-1 (450 mg) in 25% HBr—AcOH (3 mL) was added bromine (0.104 mL, 2.01 mmol) via a syringe at room temperature. After 1.5 h, the solvent was removed by nitrogen flow to give the crude product (yellow solid), which was triturated with IPE to give the titled compound (789 mg, quant, chemical purity 80%) as a slightly yellow solid.

¹H-NMR (270 MHz, CDCl₃): delta 9.35 (d, J=5.3 Hz, 1H), 8.19 (d, J=7.9 Hz, 2H), 8.06 (d, I=5.3 Hz, 1H), 7.91 (d, J=7.9 Hz, 1H), 5.05 (s, 2H), 2.45 (s, 3H). MS (ESI) m/z: 291.0 (M+H)⁺.

### EXAMPLES

### Example-1: 3-(2-(2,5-dimethyl-1-(5-methylisoxazol-3-yl)-1H-pyrrol-3-yl)-2-oxoethyl)-8,8-difluoro-1,3-diazaspiro[4.5]decan-2,4-dione

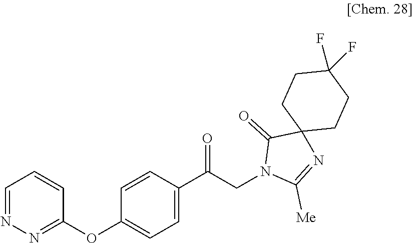 [SMILES: CC1=NC2(CCC(F)(F)CC2)C(=O)N1CC(=O)c1ccc(Oc2cccnn2)cc1]

A mixture of INT-2-1 (442 mg, 1.78 mmol), INT-1-1-A (395 mg, 1.96 mmol) and potassium carbonate (737 mg, 5.33 mmol) in DMF (20 mL) was heated at 100° C. (oil bath) for 3 h. After cooling to it, the reaction was quenched with water (15 mL) and the mixture was extracted with EtOAc-toluene (9:1)(×2). The combined solution (ca. 200 mL) was washed with water (×3), brine, dried over sodium sulfate, and filtered. The filtrate was concentrated in vacuo to give the crude product (dark brown solid), which was purified by column chromatography (biotage) on silica gel (100 g) eluting with DCM-MeOH (20:1) to give the product (246 mg) as a brown solid. The obtained solid was recrystallized from MeOH-IPE to give the titled compound (205.9 mg, 28% yield) as a slightly brown solid.

¹H-NMR (270 MHz, DMSO-d₆): delta 9.12-9.04 (m, 1H), 8.16 (d, J=8.6 Hz, 2H), 7.90-7.80 (m, 1H), 7.64-7.56 (m, 1H), 7.43 (d, J=8.6 Hz, 2H), 5.20 (s, 2H), 2.25-2.00 (m, 4H), 2.09 (s, 3H), 1.92-1.75 (m, 2H), 1.66-1.52 (m, 2H).

MS (ESI) m/z: 415.1 (M+H)⁺.

### Example-2: 2-methyl-3-(2-(4-(2-methyl-1H-benzo[d]imidazol-1-yl)phenyl)-2-oxoethyl)-1,3-diazaspiro[4.5]dec-1-en-4-one

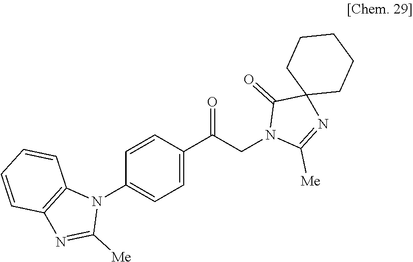 [SMILES: CC1=NC2(CCCCC2)C(=O)N1CC(=O)c1ccc(-n2c(C)nc3ccccc32)cc1]

A mixture of INT-1-1-A (20.3 mg, 0.122 mmol), INT-2-2 (50 mg, 0.122 mmol) and potassium carbonate (50.6 mg, 0.366 mmol) in DMF (1 mL) was irradiated in a microwave reactor (Biotage Initiator) at 160° C. for 20 min. The mixture was diluted with water and extracted with DCM. The combined solution was dried over sodium sulfate, filtered and concentrated in vacuo. The residual orange oil was purified by column chromatography (Biotage) on silica gel (10 g) eluting with 0-100% EtOAc in DCM, then 20% MeOH in EtOAc to give the product (14 mg, brown oil), which was triturated with hexane to give the titled compound (11 mg, 22% yield) as a brown solid.

¹H-NMR (270 MHz, DMSO-d₆): delta 8.30 (d, J=8.6 Hz, 2H), 7.81 (d, J=8.6 Hz, 21H), 7.70-7.60 (m, 1H), 7.30-7.15 (m, 3H), 5.23 (s, 2H), 2.08 (s, 3H), 1.76-1.30 (m, 10H), 1.24 (s, 3H).

MS (ESI) m/z: 415.32 (M+H)⁺.

The following Examples (3 to 6) were prepared according to the procedure of Example 1 from the intermediates (INT-2-1 to INT-2-4) and the imidazolinone derivatives (INT-1-2-A and INT-1-3-A). The further purification was carried out by preparative LC-MS system in the usual manner. The retention time and observed MS by HPLC-QC method were summarized in Table 10.

TABLE 10

| | | imidazolinone | observed | tR/ |
|---|---|---|---|---|
| Examples | alpha-haloketones | derivative | MS | method |
| 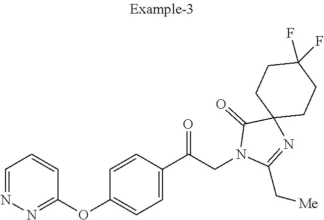 [SMILES: CCC1=NC2(CCC(F)(F)CC2)C(=O)N1CC(=O)c1ccc(Oc2cccnn2)cc1] | 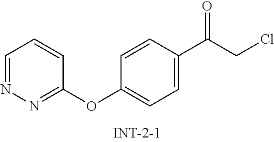 [SMILES: O=C(CCl)c1ccc(Oc2cccnn2)cc1] | 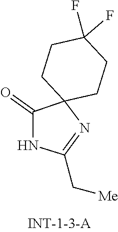 [SMILES: CCC1=NC2(CCC(F)(F)CC2)C(=O)N1] | 429.2 | 1.47 min. (QC1) |
| 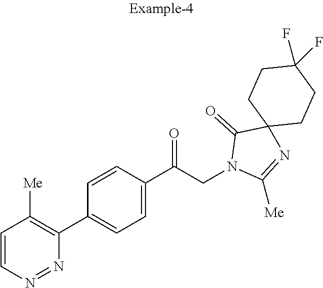 [SMILES: CC1=NC2(CCC(F)(F)CC2)C(=O)N1CC(=O)c1ccc(-c2nnccc2C)cc1] | 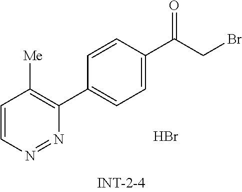 [SMILES: Cc1ccnnc1-c1ccc(C(=O)CBr)cc1 ; Br] | 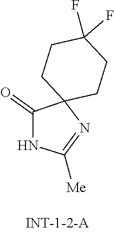 [SMILES: CC1=NC2(CCC(F)(F)CC2)C(=O)N1] | 413.2 | 1.34 min. (QC1) |
| 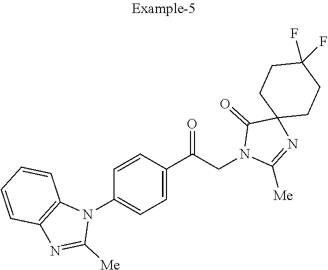 [SMILES: CC1=NC2(CCC(F)(F)CC2)C(=O)N1CC(=O)c1ccc(-n2c(C)nc3ccccc32)cc1] | 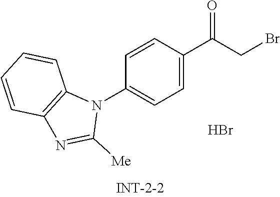 [SMILES: Cc1nc2ccccc2n1-c1ccc(C(=O)CBr)cc1 ; Br] | 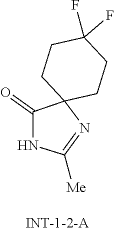 [SMILES: CC1=NC2(CCC(F)(F)CC2)C(=O)N1] | 449.5 | 1.52 min. (QC1) |
| 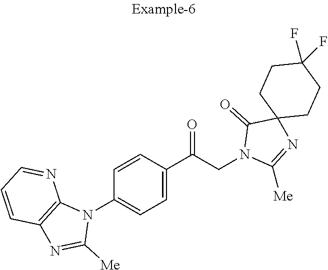 [SMILES: CC1=NC2(CCC(F)(F)CC2)C(=O)N1CC(=O)c1ccc(-n2c(C)nc3cccnc32)cc1] | 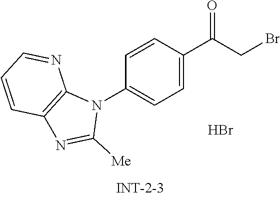 [SMILES: Cc1nc2cccnc2n1-c1ccc(C(=O)CBr)cc1 ; Br] | 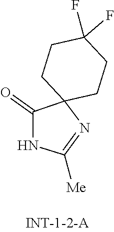 [SMILES: CC1=NC2(CCC(F)(F)CC2)C(=O)N1] | 452.2 | 1.37 min. (QC1) |

TABLE 11

| Examples | Structure | ¹H-NMR Data |
|---|---|---|
| Example-5 | 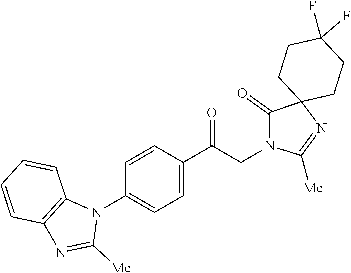 [SMILES: CC1=NC2(CCC(F)(F)CC2)C(=O)N1CC(=O)c1ccc(-n2c(C)nc3ccccc32)cc1] | ¹H-NMR (600 MHz, DMSO-d₆): delta 8.31 (d, J = 8.4 Hz, 2H), 7.82 (d, J = 8.4 Hz, 2H), 7.66 (d, J = 7.7 Hz, 1H), 7.28-7.19 (m, 3H), 5.27 (s, 2H), 2.50 (s, 3H), 2.22-2.10 (m, 4H), 2.12 (s, 3H), 1.89-1.81 (m, 2H), 1.65-1.57 (m, 2H). |

Measurement of the Menthol-Induced Ca²⁺ Influx in HEK293 Cells Stably Expressing Human TRPM8

A cell-based Ca²⁺ influx assay using HEK293 cells stably expressing human TRPM8 is used to identify the activity of compounds.

HEK293 cells stably expressing human TRPM8 are grown in T175 flasks at 37° C. in a 5% CO₂ humidified incubator to about 80% confluence. Media composition consists of Dulbecco's Modified Eagle Medium (high glucose), 10% fetal calf serum (FCS), 100 units/mL Penicillin, 100 microg/mL Streptomycin and 600 microg/mL Geneticine. At 24 hours prior to assay, cells are seeded in poly-D-lysine coated 384-well plates (BD FALCON) at a density of 30,000 cells per well in culture medium and grown overnight in 5% CO₂ at 37° C. On the assay day, growth media is removed and cells are loaded with 0.5 microM Fluo4-AM (Molecular Probes) and 0.005% Pluronic F-127 dissolved in assay buffer (Hank's balanced salt solution (HBSS), 19.4 mM HEPES pH 7.4, 2.5 mM Probenecid) for 1 hour at room temperature. After washing with assay buffer, the cells are preincubated with various concentrations of the compounds for 5 min. The changes in intracellular calcium concentration by addition of 30 microM menthol are monitored by the cell imaging technology by Hamamatsu Photonics Functional Drug Screening System (FDSS).

The IC₅₀ values for compounds of the present invention are determined from 11-point dose-response studies. Curves are generated using the average of duplicate wells for each data point. Finally, the IC₅₀ values are calculated with the best-fit dose curve determined by XLfit (ID Business Solutions Ltd.).

All tested compounds show less than about 3 microM of IC₅₀ against TRPM8 in the above assays. Preferable compounds show less than about 500 nM of IC₅₀ against TRPM8 in the above assays. More preferable compounds show less than about 100 nM of IC₅₀ against TRPM8 in the above assays. Most preferable compounds show less than about 50 nM of IC₅₀ against TRPM8 in the above assays.

Compounds with IC₅₀ against TRPM8<500 nM are: Example 1, Example 2, Example 3, Example 4, Example 5, and Example 6.

Compounds with IC₅₀ against TRPM8<100 nM are: Example 1, Example 2, Example 3, Example 4, Example 5, and Example 6.

Compounds with IC₅₀ against TRPM8<50 nM are: Example 1, Example 2, Example 3, Example 5, and Example 6.

Measurement of the Menthol-Induced Ca Influx in a Human Malignant Melanoma Cell Lines

Since TRPM8 is expressed in a human malignant melanoma cell lines, G-361 (Health Science Research Resources Bank, Osaka, Japan), the G-361 cells are used for in vitro functional assay.

G-361 cells are grown in T175 flasks at 37*C in a 5% CO₂ humidified incubator to about 80% confluence. Media composition consists of McCoy's 5A medium and 10% FCS. At 48 hours prior to assay, cells are seeded in poly-D-lysine coated 96-well plates (Corning) at a density of 12,000 cells per well in culture medium and grown in 5% CO₂ at 37° C. On the assay day, growth media is removed and cells are loaded with 5 microM Fluo-4 AM (Molecular Probes) and 0.005% Pluronic F-127 dissolved in assay buffer (HBSS, 19.4 mM HEPES pH 7.4, 2.5 mM Probenecid) for 1 hour at room temperature. After washing with assay buffer, the cells are preincubated with various concentrations of the compounds for 5 min. The changes in intracellular calcium concentration by addition of 300 microM menthol are monitored by FDSS.

The IC₅₀ values for compounds of the present invention are determined from dose-response studies. Curves are generated using the average of duplicate wells for each data point. Finally, the IC₅₀ values are calculated with the best-fit dose curve determined by XLfit (ID Business Solutions Ltd.).

Compounds of this invention show good IC₅₀ values, which show the abovementioned practical use.

Chronic Constriction Injury (CCI)-Induced Model of Neuropathic Pain; Cold Allodynia

Male Sprague Dawley rats (7 weeks old at the start of experiment, n=7-10/treatment) purchased from Charles River Japan, Inc. are used. The CCI is made according to the method of Bennett G J and Xie Y K (Pain 1988, 33: 87-107). Rats are anesthetized with intraperitoneal injection of sodium pentobarbital. The left common sciatic nerve is exposed at the level of the middle of the thigh and four ligatures are loosely tided around it by using 4-0 silk thread (Ethicon Inc.) with about 1 mm space. Sham operation is performed in the same manner except of sciatic nerve ligation. One to two weeks following CCI surgery, cold allodynia is assessed using a cold plate (LHP-1700CP, TECA) with a temperature controller (Model 3300-0, CAL Controls Inc.) as described by Tanimoto-Mori S et al. (Behav Pharmacol., 19: 85-90, 2008). The animals are habituated to the apparatus which consists of a transparent acrylic box (10×12×12 cm) on a stainless-steel plate (15×33 cm). The surface of the cold plate held on 10° C. and the temperature of the plate is monitored continuously with a precision of 0.1° C. For testing, the rat is placed on the cold plate and the paw withdrawal latency (PWL) is measured before and after the compound administration, with a cut-off value of 120 seconds. The compounds of the invention or their vehicles are administered perorally, subcutaneously or intraperitoneally. The percentages of inhibition are calculated as follows;

$$Inhibition ( \% ) = \frac{{PWL}_{ drug} - {PWL}_{ vehicle}}{{PWL}_{ sham} - {PWL}_{ vehicle}} \times 100. \qquad [ Math . 1 ]$$

Compounds of this invention show potent activities in this model, which show the above-mentioned practical use.

Chronic Constriction Injury (CCI)-Induced Model of Neuropathic Pain; Static Allodynia

Male Sprague Dawley rats (7 weeks old at the start of experiment, n=7-10/treatment) purchased from Charles River Japan, Inc. are used. The CCI is made according to the method of Bennett G J and Xie Y K (Pain 1988, 33: 87-107). Rats are anesthetized with intraperitoneal injection of sodium pentobarbital. The left common sciatic nerve is exposed at the level of the middle of the thigh and four ligatures are loosely tided around it by using 4-0 silk thread (Ethicon Inc.) with about 1 mm space. Sham operation is performed in the same manner except of sciatic nerve ligation. Static allodynia is assessed using von Frey hairs (VFHs) at two to three weeks following CCI surgery as described by Field M J et al. (Pain 1999, 83: 303-311). The animals are habituated to grid bottom cages prior to the start of experiment. VFHs in ascending order of force (0.16, 0.4, 0.6, 1, 1.4, 2, 4, 6, 8, 10, 15 and 26 gram) are applied to the plantar surface of the hind paw. Each VFH is applied to the ipsilateral paw for 6 seconds or until a withdrawal response is occurred. Once a withdrawal response is happened, the paw is re-tested, starting with the next descending VFH until no response is occurred. The lowest amount of force required to elicit a response is recorded as paw withdrawal threshold (PWT). Static allodynia is defined as present if animals responded to or below the innocuous 1.4 gram VFH. The compounds of the invention or their vehicles are administered perorally, subcutaneously or intraperitoneally. The percentages of inhibition are calculated as follows;

$$Inhibition ( \% ) = \frac{{log}_{10} ( {PWT}_{drug} ) - {log}_{10} ( {PWT}_{vehicle} )}{{log}_{10} ( {PWT}_{sham} ) - {log}_{10} ( {PWT}_{vehicle} )} \times 100. \qquad [ Math . 2 ]$$

Compounds of this invention show potent activities in this model, which show the above-mentioned practical use.

Oxaliplatin-induced model of neuropathic pain; cold and static allodynia

Male Sprague Dawley rats (7 weeks old at the start of experiment, n=7-10/treatment) purchased from Charles River Japan, Inc. are used. The study is conducted according to the method of Gauchan P et al. (NeuroSci Lett, 2009, 458, 93-95). Oxaliplatin (Yakult Co., Ltd.) is dissolved in 5% glucose. Oxaliplatin (4 mg/kg) is injected intraperitoneally twice a week for two-week. Cold allodynia is assessed using a cold plate (LHP-1700CP, TECA) with a temperature controller (Mode13300-0, CAL Controls Inc.) as described by Tanimoto-Mori S et al. (Behav Pharmacol., 19: 85-90, 2008). The animals are habituated to the apparatus which consists of a transparent acrylic box (10×12×12 cm) on a stainless-steel plate (15×33 cm). The surface of the cold plate held on 10° C. and the temperature of the plate is monitored continuously with a precision of 0.1° C. For testing, the animal is placed on the cold plate and PWL is measured before and after the compound administration, with a cutoff value of 120 seconds. Static allodynia is assessed using VFHs. The animals are habituated to grid or mesh bottom cages prior to the start of experiment VFHs in ascending order of force (0.16, 0.4, 0.6, 1, 1.4, 2, 4, 6, 8, 10, 15 and 26 gram) are applied to the plantar surface of the hind paw. Once a withdrawal response is happened, the paw is re-tested, starting with the next descending VFH until no response is occurred. The lowest amount of force required to elicit a response is recorded as paw withdrawal threshold (PWT). For testing, PWT is measured before and after the compound administration. The compounds of the invention or their vehicles are administered perorally, subcutaneously or intraperitoneally.

Compounds of this invention show potent activities in this model, which show the above-mentioned practical use.

Oxaliplatin-Induced Model of Neuropathic Pain; Cold Hyperalgesia/Allodynia

Male Sprague Dawley rats (7 weeks old, n=8-10/treatment) purchased from Charles River Japan, Inc. were used. Oxaliplatin (Wako Pure Chemical Industries, Led.) was dissolved in 5% glucose for injection to make 4 mg/mL solution. Oxaliplatin (4 mg/kg) was injected intraperitoneally twice a week for two-week (on Days 1, 2, 8 and 9) in a volume of 1 mL/kg. First day of treatment was defined as Day 1. Cold hyperalgesia/allodynia was assessed by acetone test. The animals were habituated to grid or mesh bottom cages prior to the start of experiment. Acetone (50 mL) was applied to the plantar surface of the hind paw. After the application, nociceptive responses were scored as follows: 0; no response, 1; stamping and/or lifting of the paw, 2; licking/biting or flinching of the paw once, 3; repeated licking/biting and/or flinching of the paw. Acetone was repeatedly applied to the left and right hind paws (twice for each, total 4 applications), thus total score were maximum 12 and minimum 0. For testing, total score was measured before and after the compound administration. The compounds of the invention or their vehicles were administered perorally, subcutaneously or intraperitoneally.

Compounds of this invention showed potent activities in this model, which show the above-mentioned practical use.

Icilin-Induced Wet-Dog Shakes in Rats

Male Sprague Dawley rats (6-7 weeks old, Charles River Japan, Inc., n=5-8/treatment) are used to evaluate the ability of the compounds of the invention to block the spontaneous wet-dog shakes (WDS) behavior induced by icilin. Rats are acclimated in observation boxes (21.5×26.5×25.0 cm) for at least 20 minutes before icilin injection. Icilin (Sigma) dissolved in PEG400 is administered intraperitoneally at 0.5, 1.0 or 2.5 mg/kg and spontaneous WDS are counted over 30 min post-icilin. The compounds of the invention or their vehicles are administered perorally, subcutaneously or intraperitoneally before icilin injection. The percentages of inhibition are calculated as follows;

% inhibition−[1−(compound WDS count/vehicle WDS count)]×100. [Math.3]

Compounds of this invention show potent activities in this model, which show the above-mentioned practical use.

Measurement of the Micturition Frequency in Guinea Pigs In Vivo

Female Guinea Pigs (300-450 g) are anaesthetized with urethane. A midline abdominal incision is performed, both ureters are exposed and ligated, a catheter is implanted in the bladder pole and the abdomen is closed. For administration of the compounds the vena jugularis is exposed and cannulated with a catheter. After this surgery, the bladder catheter is connected via a t-shaped tube to an infusion pump and to a pressure transducer. Saline is infused and intrabladder pressure is registered. After 1 h of equilibration period and the establishment of constant voiding cycles, menthol (0.2-0.6 mM) is added to the infused saline. At this point also vehicle (control group) or TRPM8 antagonists are administered i.v. as bolus injection. The effect of treatment on the micturition interval (corresponding to bladder capacity) and micturition pressure is calculated and compared between vehicle-treated and compound-treated groups.

Compounds of this invention show potent activities in this model, which show the above-mentioned practical use.

Measurement of Over Active Bladder in Anesthetized Cystitis Rats

Female Sprague-Dawley rats (7-8 weeks old, Japan SLC) are used. Cyclophosphamide (Wako) dissolved in saline (Otauka) is administered intraperitoneally at 200 mg/kg. On the next day, rats are anesthetized by administration of urethane at 0.9 mg/kg, s.c. The abdomen is opened through a midline incision, and a polyethylene catheter is implanted into the bladder through the dome. The bladder catheter is connected via T-tube to a pressure transducer and a microinjection pump. Saline is infused at room temperature into the bladder at a rate of 3 mL/hour. Intravesical pressure is recorded continuously on a chart pen recorder for about 1 hour before a test compound administration.

A testing compound dissolved in PBS containing WellSolve (Celeste) is administered intravenously at 1 mg/kg, 3 mg/kg, 5 mg/kg or 10 mg/kg.

The micturition frequency calculated from micturition interval during 60 min after administration of testing compound was analyzed from the cystometry data. The testing compounds mediated inhibition of the frequency was evaluated using Dunnett method vs vehicle. A probability levels less than 5% is accepted as significant difference. Data are analyzed as this mean+/−SEM from 8-12 rats.

All tested compounds show significant effect on over active bladder in anesthetized cystitis rats.

Human Dofetilide Binding Assay

Human HERG transfected HEK293S cells are prepared and grown in-house. The collected cells are suspended in 50 mM Tris-HCl (pH 7.4 at 4° C.) and homogenized using a hand held Polytron PT 1200 disruptor set at full power for 20 sec on ice. The homogenates are centrifuged at 48,000×g at 4° C. for 20 min. The pellets are then resuspended, homogenized, and centrifuged once more in the same manner. The final pellets are resuspended in an appropriate volume of 50 mM Tris-HCl, 10 mM KCl, 1 mM MgCl₂ (pH 7.4 at 4° C.), homogenized, aliquoted and stored at −80° C. until use. An aliquot of membrane fractions is used for protein concentration determination using BCA protein assay kit (PIERCE) and ARVOsx plate reader (Wallac). Binding assays are conducted in a total volume of 30 microL in 384-well plates. The activity is measured by PHERAstar (BMG LABTECH) using fluorescence polarization technology. Test compounds (10 microL) are incubated with 10 microL of fluorescence ligand (6 nM Cy3B tagged dofetilide derivative) and 10 microL of membrane homogenate (6 microgram protein) for 120 minutes at room temperature. Nonspecific binding is determined by 10 microM E4031 at the final concentration.

All tested compounds of the invention show higher IC₅₀ values in human dofetilide binding than IC₅₀ values in TRPM8 functional assay described above.

Metabolic Stability Assay:

Half-Life in Human Liver Microsomes (HLM)

Test compounds (1 microM) are incubated with 1 mM MgCl₂ and 0.78 mg/mL HLM (HL101) or 0.74 mg/mL HLM (Gentest UltraPool 150) or 0.61 mg/mL HLM (XenoTech XTreme 200) in 100 mM potassium phosphate buffer (pH 7.4) at 37° C. on the 96-deep well plate. The reaction mixture is split into two groups, a non-P450 and a P450 group on necessary. NADPH is only added to the reaction mixture of the P450 group. (NADPH generation system is also used instead of NADPH.) An aliquot of samples of P450 group is collected at 0, 10, 30, and 60 min time point, where 0 min time point indicated the time when NADPH is added into the reaction mixture of P450 group. An aliquot of samples of non-P450 group is collected at −10 and 65 min time point. Collected aliquots are extracted with acetonitrile solution containing an internal standard. The precipitated protein is spun down in centrifuge (2000 rpm, 15 min). The compound concentration in supernatant is measured by LC/MS/MS system.

The half-life value is obtained by plotting the natural logarithm of the peak area ratio of compounds/internal standard versus time. The slope of the line of best fit through the points yield the rate of metabolism (k). This is converted to a half-life value using following equations:

Half-life=in 2/k [Math.4]

The compounds of this invention show preferable stability, which show the abovementioned practical use.

The closest compound described as an example 2-121 in WO2014/130582 has less than 5 minutes of the half-live in HLM and has the large intrinsic clearance (CLᵢₙₜ) of more than 215 mL/min/kg, whereas the present invention has more than 5 minutes in the half-live in HLM and CLᵢₙₜ of <100 mL/min/kg in metabolism stability assay, which leads to good pharmacokinetic properties.

Drug-Drug Interaction Assay

This method essentially involves determining the percent inhibition of metabolites formation from probes (tacrine 2 microM or phenacetin 50 microM for CYP1A2, bupropion 3 microM for CYP2B6, amodiaquine 2 microM for CYP2C8, diclofenac 5 or 10 microM for CYP2C9, S-mephenytoin 40 microM for CYP2C19, dextromethorphan 5 microM or bufuralol 5 microM for CYP2D6, and midazolam 2 microM or 2.5 microM for CYP3A4) at 3 microM or 0.4-50 microM of the each compound.

More specifically, the assay is carried out as follows. The compounds (60 microM, 10 microL) are pre-incubated in 170 microL of mixture including 0.1 mg protein/mL or 0.05 mg protein/mL human liver microsomes, 100 mM potassium phosphate buffer (pH 7.4), 1 mM MgCl₂ or 3.3 mM MgCl₂ and probes as substrate for appropriate time (5 min or 30 min). Reaction is started by adding a 20 microL of 10 mM NADPH or 10 microL of 13 microM NADPH. The assay plate is incubated at 37° C. Acetonitrile or methanol is added to the incubate solution at appropriate time (8 min or 10 min).

The metabolites' concentration in the supernatant is measured by LC/MS/MS system.

The degree of drug-drug interaction is interpreted based on generation % of metabolites in the presence or absence of test compound or IC₅₀ values calculated from generation % of metabolism vs. compound concentration.

The compounds of this invention show preferable results, which show the abovementioned practical use.

Plasma Protein Binding Assay

Plasma protein binding of the test compound (1 microM) is measured by the method of equilibrium dialysis using 96-well plate type equipment. HTD96a (registered trademark), regenerated cellulose membranes (molecular weight cut-off 12,000-14,000, 22 mm×120 mm) are soaked for over night in distilled water, then for 15 minutes in 30% ethanol, and finally for 20 minutes in dialysis buffer (Dulbecco's phosphate buffered saline, minus CaCl₂ and MgCl₂). Frozen plasma of human, Sprague-Dawley rats, and Beagle dogs are used. The dialysis equipment is assembled and added 150 microL of compound-fortified plasma to one side of each well and 150 microL of dialysis buffer to the other side of each well. After 4 hours incubation at 37° C. for 150 rpm, aliquots of plasma and buffer are sampled. The compound in plasma and buffer are extracted with 300 microL of acetonitrile or acetonitrile/methanol (1/1) containing internal standard compounds for analysis. The concentration of the compound is determined with LC/MS/MS analysis.

The fraction of the compound unbound is calculated by the following equation (A) or (B):

[Math.5]

fu=1−[{(plasma]_{eq}−[buffer]_{eq})/([plasma]_{eq})} (A)

wherein [plasma]_{eq} and [buffer]_{eq} are the concentrations of the compound in plasma and buffer, respectively.

$$( B ) fu ( \% ) = \frac{Cb \text{/} Cis , b \times 4}{Cp \text{/} Cis , p \times 4 / 3} \times 100 \qquad [ Math . 6 ]$$

wherein Cp is the peak area of the compound in plasma sample;

Cis.p is the peak area of the internal standard in plasma sample;

Cb is the peak area of the compound in buffer sample;

Cis,b is the peak area of the internal standard in buffer sample; and

4 and 4/3 is the reciprocal of the dilution rate in plasma and buffer, respectively.

The compounds of this invention show preferable plasma protein binding, which show the above-mentioned practical use.

Equilibrium Aqueous Solubility Study

The DMSO solution (2 microL, 30 mM) of each compound is dispensed into each well of a 96-well glass bottom plate. Potassium phosphate buffer solution (50 mM, 198 microL, pH 6.5) is added to each well, and the mixture is incubated at 37° C. with rotate shaking for 24 hours. After centrifugation at 2000 g for 5 minutes, the supernatant is filtered through the polycarbonate Isopore membrane. The concentration of samples is determined by a general gradient HPLC method (J. Pharm. Sci., 95, 2115-2122, 2006).

All publications, including but not limited to, issued patents, patent applications, and journal articles, cited in this application are each herein incorporated by reference in their entirety. Although the invention has been described above with reference to the disclosed embodiments, those skilled in the art would readily appreciate that the specific experiments detailed are only illustrative of the invention. It should be understood that various modifications can be made without departing from the spirit of the invention. Accordingly, the invention is limited only by the following claims.

1. A compound of the following formula (I) 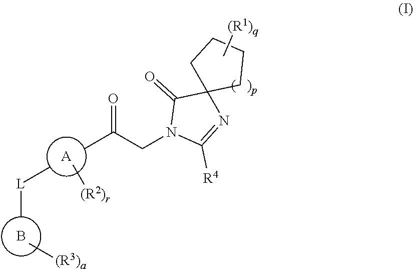
wherein
A is aryl and heteroaryl;
B is aryl and heteroaryl;
L is independently selected from the group consisting of a chemical bond, oxygen, sulfur, —NR⁵—, —(CR^{A}R^{B})ₜ—, —O(CR^{A}R^{B})ₜ—, —(CR^{A}R^{B})ₜO—, —N(R⁵)(CR^{A}R^{B})ₜ—, —(CR^{A}R^{B})ₜN(R⁵)—, —N(R⁵)(CR^{A}R^{B})ₜO—, and —O(CR^{A}R^{B})ₜN(R⁵)—;
R^{A} and R^{B} are independently selected from the group consisting of (1) hydrogen, (2) halogen, (3) (C₁-C₁₀)alkyl, (4) (C₃-C₁₀)cycloalkyl and (5) (C₁-C₁₀)haloalkyl; or R^{A} and R^{B} may form a 3 to 8 membered ring which may contain one or more heteroatoms independently selected from oxygen, sulfur and nitrogen; and said ring is optionally substituted with 1 to 6 substituents independently selected from (1) hydrogen, (2) halogen, (3) hydroxy, (4) (C₁-C₁₀)alkyl, (5) (C₃-C₁₀)cycloalky, (6) (C₁-C₁₀)haloalkyl, (7) (C₁-C₁₀)alkoxy and (8) (C₁-C₁₀)haloalkoxy;
R¹ is independently selected from the group consisting of (1) hydrogen, (2) halogen, (3) amino, (4) cyano, (5) hydroxyl, (6) (C₁-C₁₀)alkyl, (7) (C₃-C₁₀)cycloalkyl, (8) (C₁-C₁₀)haloalkyl, (9) (C₁-C₁₀)alkoxy and (10) (C₁-C₁₀)haloalkoxy; two R′ on the same carbon or the different carbons are possible to form a 3 to 8 membered ring which may contain an atom selected from oxygen, sulfur and nitrogen; and said ring is optionally substituted with 1 to 6 substituents independently selected from (1) hydrogen, (2) halogen, (3) hydroxy, (4) (C₁-C₁₀)alkyl, (5) (C₃-C₁₀)cycloalkyl, (6) (C₁-C₁₀)haloalkyl, (7) (C₁-C₁₀)alkoxy, and (8) (C₁-C₁₀)haloalkoxy;
R² is independently selected from the group consisting of (1) hydrogen, (2) halogen, (3) amino, (4) —NH(C₁-C₆)alkyl, (5) —N[(C₁-C₆)alkyl]₂ wherein the alkyl is same or different, (6) cyano, (7) hydroxyl, (8) nitro, (9) (C₁-C₆)alkylthio, (10) (C₁-C₁₀)alkyl, (11) (C₃-C₁₀)cycloalkyl, (12) (C₁-C₁₀)alkoxy, (13) (C₁-C₁₀)haloalkyl and (14) (C₁-C₁₀)haloalkoxy;
R³ is independently selected from the group consisting of (1) hydrogen, (2) halogen, (3) cyano, (4) nitro, (5) hydroxyl, (6) (C₁-C₆)alkylthio, (7) (C₁-C₆)alkylsulfinyl, (8) (C₁-C₆)alkylsulfonyl, (9) —NR⁶R⁷, (10) —C(═O)NR⁶R⁷, (11) tri(C₁-C₆)alkylsilyl, (12) (C₁-C₁₀)alkyl, (13) (C₃-C₁₀)cycloalkyl, (14) (C₃-C₆)alkoxy(C₀-C₆)alkyl, (15) (C₃-C₁₀)cycloalkoxy, (16) —C(═O)(C₁-C₆)alkyl, (17) —C(═O)O(C₁-C₆)alkyl and (18) —C(═O)OH; said (C₁-C₁₀)alkyl, (C₃-C₁₀)cycloalkyl, (C₁-C₆)alkoxy(C₀-C₆)alkyl and (C₃-C₁₀)cycloalkoxy are optionally substituted with 1 to 6 substituents independently
selected from (1) hydrogen, (2) halogen, (3) hydroxyl, (4) cyano, (5) (C₃-C₁₀)cycloalkyl, (6) (C₁-C₁₀)haloalkyl, (7) (C₁-C₁₀)alkoxy, (8) (C₁-C₁₀)haloalkoxy and (9) —NR⁶R⁷;
wherein R⁶ and R⁷, together with nitrogen atom to which they are attached, may form a 3 to 10 membered ring which may contain an atom selected from oxygen, sulfur and nitrogen; and said ring is optionally substituted with 1 to 6 substituents independently selected from (1) hydrogen, (2) halogen, (3) hydroxyl, (4) (C₁-C₁₀)alkyl, (5) (C₃-C₁₀)cycloalkyl, (6) (C₁-C₁₀)haloalkyl, (7) (C₁-C₁₀)alkoxy and (8) (C₁-C₁₀)haloalkoxy;
R⁴ is independently selected from the group consisting of (1) hydrogen, (2) (C₁-C₁₀)alkyl, (3) (C₃-C₁₀)cycloalkyl and (4) (C₁-C₁₀)haloalkyl;
R⁵, R⁶ and R⁷ are independently selected from the group consisting of (1) hydrogen, (2) (C₁-C₁₀)alkyl, (3) (C₃-C₁₀)cycloalkyl, (4) (C₁-C₁₀)haloalkyl, (5) hydroxyl(C₁-C₁₀)alkyl, (6) (C₁-C₁₀)alkoxy(C₁-C₁₀)alkyl, (7) H2N—(C₁-C₁₀)alkyl, (8) [(C₁-C₁₀)alkyl]NH—(C₁-C₁₀)alkyl, (9) [(C₁-C₁₀)alkyl]₂N—(C₁-C₁₀)alkyl, (10) (C₁-C₁₀)alkylcarbonyl and (11) (C₁-C₁₀)alkylsulfonyl;
p is 1, 2, 3 or 4;
q is 1, 2, 3 or 4; when q is two or more than two, R¹ is same or different,
r is 1, 2, 3 or 4; when r is two or more than two, R² is same or different,
s is 1, 2, 3, 4, 5, 6 or 7; when s is two or more than two, R³ is same or different,
t is 1, 2 or 3; when t is two or more than two, R^{A} and R^{B} are same or different,
or a pharmaceutically acceptable salt thereof or a prodrug thereof.

2. The compound described in claim 1 wherein
A is 6 membered aryl or 5 to 6 membered heteroaryl
or a pharmaceutically acceptable salt thereof or a prodrug thereof.

3. The compound described in claim 1 wherein
A is independently selected from the group consisting of
benzene, pyridine, pyridazine, pyrazine, pyrimidine, triazine, thiophene, furan, pyrrole, imidazole, pyrazole, thiazole, isothiazole, oxazole, isoxazole, and triazole.
or a pharmaceutically acceptable salt thereof or a prodrug thereof.

4. The compound as claimed in claim 1 which is selected from:
8,8-difluoro-2-methyl-3-(2-oxo-2-(4-(pyridazin-3-yloxy)phenyl)ethyl)-1,3-diazaspiro[4.5]dec-1-en-4-one;
2-methyl-3-(2-(4-(2-methyl-1H-benzo[d]imidazol-1-yl)phenyl)-2-oxoethyl)-1,3-diazaspiro[4.5]dec-1-en-4-one;
2-ethyl-8,8-difluoro-3-(2-oxo-2-(4-(pyridazin-3-yloxy)phenyl)ethyl)-1,3-diazaspiro[4.5]dec-1-en-4-one;
8,8-difluoro-2-methyl-3-(2-(4-(4-methylpyridazin-3-yl)phenyl)-2-oxoethyl)-1,3-diazaspiro[4.5]dec-1-en-4-one;
8,8-difluoro-2-methyl-3-(2-(4-(2-methyl-1H-benzo[d]imidazol-1-yl)phenyl)-2-oxoethyl)-1,3-diazaspiro[4.5]dec-1-en-4-one;
8,8-difluoro-2-methyl-3-(2-(4-(2-methyl-3H-imidazo[4,5-b]pyridin-3-yl)phenyl)-2-oxoethyl)-1,3-diazaspiro[4.5]dec-1-en-4-one;
or a pharmaceutically acceptable salt thereof or a prodrug thereof.

5-6. (canceled)

7. A method for the treatment of a condition or disorder mediated by TRPM8 receptor antagonistic activity in a mammalian subject, including a human, which comprises administering to a mammal in need of such treatment a therapeutically effective amount of a compound described in claim 1 or a pharmaceutically acceptable salt thereof or a prodrug thereof.

8. The method as claimed in claim 7, wherein the condition or disorder is one or more of inflammatory, pain and urological diseases or disorders, including chronic pain; neuropathic pain including cold allodynia and diabetic neuropathy; postoperative pain; osteoarthritis; rheumatoid arthritic pain; cancer pain; neuralgia; neuropathies; algesia; dentin hypersensitivity; nerve injury; migraine; cluster and tension headache; ischaemia; irritable bowel syndrome; Raynaud's syndrome; neurodegeneration; fibromyalgia; stroke; itch; psychiatric disorders including anxiety and depression; inflammatory disorders including asthma, chronic obstructive pulmonary, airways disease including COPD, pulmonary hypertension; anxiety including other stress-related disorders; and urological diseases or disorders including detrusor overactivity or overactive bladder, urinary incontinence, neurogenic detrusor overactivity or detrusor hyperreflexia, idiopathic detrusor overactivity or detrusor instability, benign prostatic hyperplasia, and lower urinary tract symptoms; and combinations thereof.

9. A pharmaceutical composition comprising a compound or a pharmaceutically acceptable salt thereof or a prodrug thereof, as described in claim 1, and a pharmaceutically acceptable carrier.

10. The pharmaceutical composition as claimed in claim 9, further comprising another pharmacologically active agent.

11. (canceled)

12. A process for preparing a pharmaceutical composition, wherein the process comprises mixing a compound described in claim 1 or a pharmaceutically acceptable salt thereof or a prodrug thereof and a pharmaceutically acceptable carrier or excipient.
